(19) United States
(12) Patent Application Publication
(10) Pub. No.: US 2026/0049359 A1
(43) Pub. Date: Feb. 19, 2026

(54) PREDICTION OF RESPONSE TO IMMUNE THERAPY IN BREAST CANCER PATIENTS

(71) Applicants: Agendia N.V., Amsterdam (NL); The Regents of the University of California, Oakland, CA (US)

(72) Inventors: Annuska Maria GLAS, Assendelft (NL); Lorenza MITTEMPERGHER, Abcoude (NL); Andrei BARCARU, Almere (NL); Midas Mees KUILMAN, Purmerend (NL); Laura Johanna VAN ’T VEER, San Francisco, CA (US); Denise WOLF, Berkeley, CA (US); Hoi Sze Christina YAU, Novato, CA (US); Laura Jane ESSERMAN, San Francisco, CA (US)

(21) Appl. No.: 18/867,386

(22) PCT Filed: May 19, 2023
(86) PCT No.: PCT/NL2023/050285

§ 371 Date: Nov. 19, 2024

(30) Foreign Application Priority Data
May 19, 2022 (EP) 22174444.4

(51) Int. Cl. C12Q 1/6886; C12Q 1/6806; G16B 25/10

(52) U.S. Cl. CPC C12Q 1/6886; C12Q 1/6806; G16B 25/10; C12Q 2600/106; C12Q 2600/158

(57) ABSTRACT

The invention relates to methods for typing a sample comprising breast cancer cells or comprising gene expression products from breast cancer cells, of an individual with breast cancer. The invention further relates to methods of treating an individual with breast cancer, that is typed according to the methods of the invention.

This application is the U.S. National Phase of International Patent Application Number PCT/NL2023/050285 filed 19 May 2023, which claims priority from EP 21174444.4 filed 19 May 2022, each of which is incorporated herein by reference.

### INCORPORATION OF SEQUENCE LISTING

A computer-readable XML file entitled “Substitute_Sequence_Listing_294-657_PCTUS_ST26,” created on Feb. 25, 2025 with a file size of 72,693 bytes contains the Sequence Listing for this application, which has been filed with this application, and is hereby incorporated by reference in its entirety.

### FIELD OF INVENTION

The invention relates to methods for typing of cancer, especially breast cancer. The invention is directed to a set of marker genes to predict response to cancer therapy.

### 1 BACKGROUND

Cancer is a leading cause of death worldwide and was responsible for nearly 10 million deaths in 2020. The most common cancer in 2020 in terms of new cases was breast cancer with 2.26 million cases and responsible for 685,000 deaths globally that year (Ferlay et al., 2021. Int J Cancer 10.1002/ijc.33588).

As in other cancers, early detection and treatment of breast cancer can effectively reduce cancer-associated mortality and significantly improve the lives of cancer patients. The overall 5-year relative survival rate of breast cancer patients is about 85%, meaning that 85% of the diagnosed breast cancer patients survive for at least five years. For patients diagnosed with early stage localized breast cancer not spread to the lymph nodes (localized) the 5-year survival rate is about 99%, while for patients diagnosed with metastasized breast cancer (distant) the 5-year survival rate is about 28% (Howlader et al. (editors). Cancer Statistics Review, 1975-2017. Table 4.13. National Cancer Institute).

Once a diagnosis of breast cancer is established and a stage of the cancer is known, an appropriate therapy can be determined. Breast cancers detected at an early stage are typically treated by surgery, often followed by radiotherapy, while metastasized cancers even if detected at an early stage, are treated systemically by chemotherapy (Maughan et al., 2010. Am Fam Physician 81:1339-1346). Adjuvant (i.e. additional) therapy is often administered as well, depending on the type of breast cancer diagnosed, to increase survival rates. As an example, for hormone receptor (HR)-positive breast cancer, adjuvant hormone-therapy is often recommended. In addition, for a large tumour that is to be removed by surgery, adjuvant chemotherapy is often administered after removal of the primary tumor (Anampa et al., 2015. BMC Med 13, 195).

Several gene signature tests have been developed to determine gene expression profiles of breast cancer samples aiming to stratify the cancer into subtypes and/or into risk groups. An example of the former is the BluePrint® test (U.S. Pat. Nos. 9,175,351; 10,072,301; Krijgsman et al., 2012. Br Can Res Treatm 133:37-47), a molecular subtyping test, analyzing the activity of 80 genes to enable stratification of a breast cancer into one of the three following subtypes: Luminal-type, HER2-type and Basal-type (Perou et al., 2000. Nature 406:747-752). An example of the latter is, the MammaPrint® (also termed “Amsterdam gene signature test” or MP) test for the stratification of breast cancer patients in Low- and High risk for developing distant metastases within 5 years after diagnosis. Extensive validation studies (Drukker et al., 2013. Int J Cancer 133:929-936; Bueno-de-Mesquita et al., 2007. Lancet Oncol 8:1079-1087; van de Vijver et al., 2002. New Engl J Med 34:1999-2009) and the recent MINDACT clinical trial (Cardoso et al., 2016. N Engl J Med 375:717-729) have demonstrated the clinical utility of MammaPrint (level 1A clinical evidence), making it an unique example of a clinical diagnostic test that may help guide physicians in treatment decisions for breast cancer patients.

Therapy that is administered before surgery is termed neoadjuvant therapy and is a commonly used therapeutic approach for breast cancer patients (Untch et al., 2014. The Breast 23:526-537). Neoadjuvant therapy improves success rates of breast-conserving surgery due to downstaging of the tumour and allows for assessment of the tumour biology which is useful to predict long-term clinical outcomes (Fisher et al., 1998. J Clin Oncol 16:2627-2685; De Mattos-Arruda et al., 2016. Nat Rev Clin Oncol 13:566-579). In particular neoadjuvant immune therapy, including treatment with immunotherapeutic agents (often in combination with a chemotherapeutic drug) before surgery, has been shown to improve the progression-free survival of breast cancer patients and is a promising new treatment tool (Schmid et al., 2018. N Engl J Med 379:2108-2121). Since not all breast cancer patients benefit from a combination with immune therapy and some even experience severe side effects, the identification of predictive markers of response is critical (Fountzila and Ignatiadis, 2020. Ecancer 14:1147).

The I-SPY 2 TRIAL (NCT01042379), sponsored by Quantum Leap Healthcare Collaborative, is a standing Phase 2 randomized, controlled, multi-center trial for women with newly diagnosed, locally advanced breast cancer (Stage II/III), and is designed to screen promising new treatments and identify which therapies are most effective in specific patient subgroups based on molecular characteristics, including biomarker signatures. The trial is an adaptive study design assessing a combination of biologically targeted investigational drugs with standard chemotherapy, compared to standard chemotherapy alone. The primary endpoint is to determine whether a combination of certain therapies increases the probability of a pathological complete response (pCR) for breast cancer patients at the time of surgery (Barker et al., 2009. Clin Pharmacol Ther 86:97-100).

In one of the treatment arms in the I-SPY 2 TRIAL immune therapy, comprising a combination of paclitaxel and pembrolizumab, is tested on HER2-negative patients with a MammaPrint High Risk profile and with either basal or luminal subtype by the BluePrint test. Paclitaxel is a well-known chemotherapeutic drug and pembrolizumab is a monoclonal antibody that targets programmed cell death protein 1 (PD-1) receptors of lymphocytes. The binding to PD-1 blocks the interaction with the PD-1 ligands (PD-L1 and PD-L2) and in this way releases the PD-1 pathway-mediated inhibition of the immune response, including the anti-tumour immune response. The addition of pembrolizumab to the standard chemotherapy resulted in a more than two-fold increase of the pCR rates compared to chemotherapy alone for both HR-positive/HER2-negative and triple negative breast cancer (Nanda et al., 2020. JAMA Oncol 6:676-684). Previous research by I-SPY 2 investigators showed that pCR to this therapy can be predicted with a microarray gene expression analysis of different marker genes (Pusztai et al., 2021. Cancer Cell in press). However, while these prediction signatures were successful with fresh frozen (FF) conserved breast cancer tissue samples, the translation for use with formalin-fixed paraffin-embedded (FFPE) tissue samples was not. For example, PD1 (PDCD1) alone as a marker has an accuracy of 0.71%.

PDL1 (CD274)) alone as a marker has an accuracy of 0.67%. Gene signatures as described in Danaher et al., 2017 (Danaher et al., 2017. J Immunother Cancer 5:18) have an accuracy of 0.59% (T cells); of 0.62% (B cells) and of 0.70% (dendritic cells), while a STAT1 signature (Chehani Alles et al., 2009. PLOS One 4: e4710) has an accuracy of 0.72%.

There is thus a need for a method to predict a response to immune therapy in breast cancer patients that is sensitive and accurate. Furthermore, such method preferably can be used on FF conserved tissue samples, as well as on FFPE conserved tissues.

### 2 SUMMARY

The invention provides a method of typing a sample comprising breast cancer cells or comprising gene expression products from breast cancer cells, of an individual who is diagnosed with breast cancer, comprising (i) isolating RNA from the sample obtained from the individual; (ii) determining an expression level of at least 16 marker genes in the isolated RNA to thereby provide an expression profile of said marker genes, wherein the marker genes are selected from the genes listed in Table 1; (iii) comparing the individual's expression profile to a reference expression profile of the at least 16 marker genes; thereby typing the sample for a response to auxiliary immune therapy.

Said sample preferably is a fresh frozen sample or a formalin-fixed, paraffin-embedded sample.

In a preferred method of the invention, an individual who is typed as positively responding to auxiliary immune therapy is treated with an immune checkpoint inhibitor such as programmed cell death protein 1 (PD1) or programmed cell death ligand 1 (PDL1) inhibitor, preferably a programmed cell death protein 1 (PD1) binding antibody. Said immune checkpoint inhibitor may be combined with a platinum-based compound and/or a taxane for treatment of said individual.

The determination of an expression profile is preferably performed using RNA-sequencing or microarray gene expression analysis.

The expression profile in preferred methods of the invention comprises at least 17 different marker genes, preferably at least 18 different marker genes, preferably at least 19 different marker genes, more preferably at least 20 different marker genes, more preferably at least 21 different marker genes, more preferably at least 22 different marker genes, more preferably at least 23 different marker genes, more preferably at least 24 different marker genes, more preferably at least 25 different marker genes, more preferably at least 26 different marker genes, more preferably at least 27 different marker genes, more preferably at least 28 different marker genes, more preferably at least 29 different marker genes, more preferably at least 30 different marker genes, more preferably at least 31 different marker genes, more preferably at least 32 different marker genes, more preferably at least 33 different marker genes, more preferably at least 34 different marker genes, more preferably at least 35 different marker genes, more preferably at least 36 different marker genes, more preferably at least 37 different marker genes, more preferably at least 38 different marker genes, more preferably at least 39 different marker genes, more preferably at least 40 different marker genes, more preferably at least 41 different marker genes, more preferably at least 42 different marker genes, more preferably at least 43 different marker genes, more preferably at least 44 different marker genes, more preferably at least 45 different marker genes, more preferably at least 46 different marker genes, more preferably at least 47 different marker genes, more preferably at least 48 different marker genes, more preferably at least 49 different marker genes, more preferably at least 50 different marker genes, more preferably at least 51 different marker genes, more preferably at least 52 different marker genes is characterised, wherein the marker genes are selected from the genes listed in Table 1.

In a most preferred method according to the invention, the expression profile of 53 different marker genes is determined, whereby the marker genes are the genes listed in Table 1.

In methods of the invention, a reference expression profile may be composed of the average expression levels of the marker genes specified in step (ii) of individuals having a positive response to auxiliary immune therapy, of individuals not having a positive response to auxiliary immune therapy, or of a mixture of individuals having a positive response to auxiliary immune therapy and individuals not having a positive response to auxiliary immune therapy.

In methods of the invention, the individual's expression profile may be compared to two reference expression profiles, wherein a first reference expression profile is composed of the average expression levels of the marker genes specified in step (ii) of individuals having a positive response to auxiliary immune therapy and a second reference expression profile is composed of the average expression level of the marker genes specified in step (ii) of individuals not having a positive response to auxiliary immune therapy.

A preferred positive response to auxiliary immune therapy is a pathologic complete response (pCR).

The invention further provides a method of treating an individual with breast cancer, comprising typing a sample from said individual using a method of typing according to the invention; treating an individual that is typed as having a positive response to auxiliary immunotherapy with auxiliary immunotherapy, optionally in combination with chemotherapy; and treating an individual that is typed as not having a positive response to auxiliary immunotherapy with chemotherapy.

Said auxiliary immunotherapy preferably comprises an immune checkpoint inhibitor, preferably a programmed cell death protein 1 (PD1) binding antibody.

In methods of treatment according to the invention, the auxiliary immunotherapy may be combined with chemotherapy such as a platinum-based compound and/or a taxane. Said platinum-based compound preferably is or comprises carboplatin. Said taxane preferably is or comprises paclitaxel.

### 3 BRIEF DESCRIPTION OF THE DRAWINGS

FIG. 1: Minimal number of genes required for satisfactory prediction (>60% accuracy). The minimal number of genes required was calculated by iteratively taking N genes randomly from all 53 genes for 10.000 iterations. From 10.000 iterations of 3 randomly selected genes over 75% had at least an accuracy of 60%, with some combinations even reaching up to 90%.

FIG. 2: Comparison of the 53-gene signature to other published immune-related signatures.

### 4 DETAILED DESCRIPTION OF THE INVENTION

### 4.1 Definitions

As is used herein, the term “cancer”, refers to a disease or disorder resulting from the proliferation of oncogenically transformed cells.

As is used herein, the term “breast cancer”, refers to a cancer originating from cells of the breasts.

As is used herein, the term “sample”, refers to any sample that can be completely or partly obtained from a cancerous growth of an individual by various means including, for example, biopsy such as needle biopsy and surgery. The term comprises any sample comprising breast cancer cells from an individual, or suspected to comprise breast cancer cells from an individual, such as a tumour or liquid biopsy. Preferably, at least 5% of the sample consists of breast cancer cells. More preferably at least 10%, 20% or 30% of the sample consists of breast cancer cells. The term “sample” further comprises any sample that may comprise gene expression products from breast cancer cells from an individual, such as blood and educated thrombocytes and/or erythrocytes.

As is used herein, the term “fresh frozen”, refers to a sample that was frozen after collection, preferably immediately frozen after collection, and conserved in frozen state thereafter.

As is used herein, the term “formalin-fixed paraffin-embedded”, refers to a tissue sample that is processed by fixation in formalin and embedding in paraffin upon collection.

As is used herein, the term “typing of a sample”, refers to the classification of a sample based on characterized features. In this invention typing includes the characterisation of expression levels of genes in a sample assisting in the prediction of a response to auxiliary immune therapy.

As is used herein, the term “response to therapy” is considered to have the same meaning as the term “response following therapy”.

As is used herein, the term “auxiliary immune therapy” refers to the inclusion of immune therapy as neoadjuvant therapy and/or as adjuvant therapy.

As is used herein, the term “individual”, refers to a human. Said individual preferably is a woman.

As is used herein, the term “primary therapy”, refers to a treatment aiming to remove a cancer, here breast cancer, from an individual, as complete as possible. Said primary therapy preferably is surgery. During surgery, axillary lymph nodes may also be removed.

As is used herein, the term “adjuvant therapy”, refers to treatment given following a primary treatment such as surgery. An aim of adjuvant therapy is, for example, to remove cancer cells that remained after primary treatment and/or to reduce the chance of recurrence of cancer cells. Adjuvant therapy in breast cancer, in addition to surgery, involves treatment including one or more of chemotherapy, radiotherapy, immune therapy, targeted therapy and hormone therapy.

As is used herein, the term “neoadjuvant therapy”, refers to treatment that is administered prior to primary treatment such as surgery. The main aim of neoadjuvant therapy in breast cancer is to render the primary treatment easier or more effective, for example by reducing the tumour size before surgery. Neoadjuvant therapy in breast cancer involves treatment including one or more of chemotherapy, radiotherapy, immune therapy, targeted therapy and hormone therapy.

As is used herein, the term “immune therapy”, or immunotherapy, refers to treatment with one or more immunotherapeutic agents that activate or suppress the immune system. In relation to auxiliary immune therapy, immune therapy includes a wide range of treatments such as immune check point inhibitors, vaccines, cytokines and monoclonal antibodies.

As is used herein, the term “immune checkpoint inhibitor”, refers to an inhibitor of an immune checkpoint molecule, a regulator of the immune system. Immune checkpoint molecules include CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328. A preferred immune checkpoint inhibitor is selective for at least one of CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328, when compared to other surface molecules, meaning that the inhibitor is at least two times more potent, preferably at least five times more potent, in inhibiting at least one of CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328, when compared to other molecules.

As is used herein, the term “Poly [ADP-ribose] polymerase (PARP) inhibitor”, refers to an inhibitor of a poly [ADP-ribose] polymerase. PARP is a key factor in the initiation of a repair response to single-strand DNA breaks (SSB). A preferred PARP inhibitor is selective for PARP1 and/or PARP2, when compared to other polymerases, meaning that the inhibitor is at least two times more potent, preferably at least five times more potent, in inhibiting PARP1 and/or PARP2, when compared to other polymerases.

As is used herein, the term “chemotherapy”, as used herein, refers to treatment with one or more chemotherapeutic agents such as alkylating agents, anthracyclines, taxanes, histone deacetylase inhibitors, topoisomerase inhibitors and platinum-based agents. Traditional chemotherapeutic agents, as used in cancer treatment, are cytotoxic and primarily kill cancer cells by inhibiting cell division.

As is used herein, the term “neoadjuvant chemotherapy”, as used herein, refers to chemotherapy that is administered prior to primary treatment such as surgery.

As is used herein, the term “pathologic complete response (pCR)”, refers to the absence of any sign of cancer in an individual with breast cancer. The term pCR may be defined as the absence of residual invasive and in situ cancer on hematoxylin and eosin evaluation of a resected breast specimen and all sampled regional lymph nodes following completion of neoadjuvant systemic therapy.

As is used herein, the term “residual disease (RD)”, refers to the presence of a sign of cancer in an individual with cancer, i.e. the absence of pCR.

As is used herein, the term “RNA”, refers to ribonucleic acid.

As is used herein, the term “isolating RNA”, refers to the extraction and purification of RNA from a biological sample. The term “isolating” refers to the removal of other components, such as proteins and DNA, at least to some extent. As is used herein, the term “gene expression level”, refers to a quantifiable level of expression of a gene of interest. A gene's expression level is often inferred by measuring a level of a gene product, such as mRNA or protein, of that gene in a sample. Said gene expression level can be determined relatively, in relation to the expression levels of other genes, such as household genes or normalization genes as described in, for example, international patent application WO2008039071; or absolutely, for example by comparing a determined level of expression to a calibration curve of the expression product of the gene.

As is used herein, the term “expression profile”, refers to the expression levels of multiple genes in a sample. An expression profile can be obtained, for example, by analysing the hybridisation pattern of a sample on a microarray, or by techniques such as RNA-sequencing or multiplex qPCR.

As is used herein, the term “marker gene”, refers to a gene whose sequence or expression level, alone or in combination with other genes, is correlated with an effect, in this application a probability of a positive or negative response to auxiliary immune therapy.

As is used herein, the term “oestrogen-receptor (ER) positive breast cancer”, refers to a breast cancer that detectably expresses oestrogen receptor (ER). ER status may be determined, for example, by IHC and/or by TargetPrint® analysis as previously reported (Roepman et al., 2009. Clin Cancer Res 15:7004-7011).

As is used herein, the term “oestrogen-receptor (ER) negative breast cancer”, refers to a breast cancer that does not detectably expresses oestrogen receptor (ER). ER status may be determined, for example, by IHC and/or by TargetPrint® analysis as previously reported (Roepman et al., 2009. Clin Cancer Res 15:7004-7011).

As is used herein, the term “human epidermal growth factor receptor 2 (HER2) negative breast cancer”, refers to a breast cancer that does not detectably express human epidermal growth factor receptor 2 (HER2). HER2 is also termed v-erb-b2 avian erythroblastic leukaemia viral oncogene homolog 2 (ERBB2) or NEU. HER2 status may be determined, for example, by immunohistochemistry, chromogenic or fluorescence in situ hybridization, and/or by TargetPrint® analysis as previously reported (Roepman et al., 2009. Clin Cancer Res 15:7004-7011).

As is used herein, the term “microarray gene expression analysis”, refers to the analysis of gene expression levels of a predefined gene set through hybridization. Microarrays, also known as chips, are microscopic slides containing microscopic spots of nucleic acid molecules from a specific gene. The nucleic acid molecules attached to the microarray act as probes for a nucleic acid molecule such as RNA or copy-DNA (cDNA) molecule, from an experimental sample. These cDNA molecules may be labelled, for example fluorescently labelled, prior to hybridization to the microarray.

The term “hybridization”, as is used herein, refers to the binding of a nucleic acid molecule such as RNA or cDNA molecule to a (partially) complementary nucleic acid probe on the microarray. Hybridization of a labelled nucleic acid molecule may result in a signal, for example a fluorescent signal, that can be detected and quantified, yielding information about the abundance of the labelled nucleic acid molecule in the experimental sample. Microarray analysis allows for the simultaneous detection of gene expression levels of a large number of genes.

As is used herein, the term “amplification”, refers to an increase in the number of copies of a particular DNA fragment through replication using at least one primer and a DNA polymerase. Known amplification methods include polymerase chain reaction (PCR) and isothermal amplification including, for example, helicase-dependent amplification (HDA) (Vincent et al., 2004. EMBO Rep 5:795-800), loop-mediated amplification (LAMP) (Notomi et al., 2000. Nucleic Acids Res 28: E63), nucleic acid sequences-based amplification (NASBA) (Guatelli et al., 1990. Proc Natl Acad Sci USA 87:1874-1878), rolling circle amplification (Ali et al., 2014. Chem Soc Rev 43:3324-3341), strand-displacement amplification (SDA) (Walker et al., 1992. Nucleic Acids Res 20:1691-6) and recombinase polymerase amplification (RPA) (Piepenburg et al., 2006. PLOS Biology 4: e204).

As is used herein, the term “RNA-Seq”, also termed “RNA-sequencing”, refers to a sequencing technique, such as a high-throughput sequencing technique, preferably using next-generation sequencing (NGS), to characterize the quantity and/or sequence of a nucleic acid molecule such as RNA in a sample. RNA-Seq can be used for gene expression analysis.

As is used herein, the term “normalisation”, refers to methods for correcting experimental variation and bias. Normalisation processes are for example important for analysis of large scale expression data, as collected using microarray or RNA-seq gene expression analysis, to preserve biological variation and eliminate experimental bias or technical variation.

As is used herein, the term “combination” refers to the administration of effective amounts of compounds to a patient in need thereof. Said compounds may be provided in one pharmaceutical preparation, or as two or more distinct pharmaceutical preparations. Said compounds may be administrated simultaneously, separately, or sequentially to each other. When administered as two or more distinct pharmaceutical preparations, they may be administered on the same day or on different days to a patient in need thereof, and using a similar or dissimilar administration protocol, e.g. daily, twice daily, biweekly, orally and/or by infusion. Said combination is preferably administered repeatedly according to a protocol that depends on the patient to be treated (age, weight, treatment history, etc.), which can be determined by a skilled physician. Said protocol may include daily administration for 1-30 days, such as 2 days, 10 days, or 21 days, followed by period of 1-14 days, such as 7 days, in which no compound is administered.

### 4.2 Sample Collection and Pre-Processing

In the invention RNA molecules are isolated from a sample comprising breast cancer cells or breast cancer derived nucleic acids of an individual with breast cancer.

The sample may be obtained from any individual with breast cancer. The individual preferably is a woman. Said individual with breast cancer can be an individual diagnosed with breast cancer or likely to be diagnosed with breast cancer. Said individual with breast cancer is an individual suffering from breast cancer or likely to suffer from breast cancer. The sample may comprise any sample comprising breast cancer cells or breast cancer derived nucleic acids from said individual such as a tumour or liquid biopsy.

The term “biopsy” refers to a biopsy derived from a primary breast cancer, while the term “liquid biopsy” refers to a biopsy obtained from a bodily fluid comprising circulating breast cancer cells or cells that have absorbed nucleic acids derived therefrom such as educated thrombocytes and/or erythrocytes (Best et al., 2015. Cancer Cell 28:666-676; Heinhuis et al., 2020. Cancers 12:1372).

Said tumour biopsy can be obtained by in numerous ways, as is known to a person skilled in the art. Preferably, the biopsy is obtained using needle biopsy or surgical biopsy. During needle biopsy, cancer cells are extracted from the breast cancer using a needle. During surgical biopsy, cells are extracted from the breast cancer after making an incision in the skin. In individuals with breast cancer, surgical biopsy is often part of the primary treatment, in which the cancer, or parts thereof, is removed from the body. It is explicitly stated that the act of removing a breast cancer, or a part of a breast cancer, from an individual is not part of this invention.

Several body fluids can potentially contain circulating breast tumour cells such as blood, plasma, serum, lymphatic fluid, saliva, faeces, urine and cerebrospinal fluid. Preferably, blood or plasma is preferably used as bodily fluid to provide a liquid biopsy of breast cancer.

The sample may be collected in any clinically acceptable manner. Said sample preferably is collected and conserved upon isolation such as to preserve at least RNA. RNA can be obtained from a sample immediately upon harvesting, or from a conserved sample. A sample can be conserved by fixation e.g. in formalin and/or by treating the sample with an RNase inhibitor, such as RNasin (Promega) and RNasecure (Invitrogen), or an RNA stabilisation agent, such as RNAlater (Invitrogen). Preferred conservation methods of samples include fresh frozen (FF) conservation, for example in dry ice or in liquid nitrogen, and formalin-fixed paraffin-embedded (FFPE) conservation.

RNA can be isolated from a sample by methods known in the art. There are three main categories of RNA extraction techniques known to date: organic extraction involving a chaotropic agent such as guanidinium thiocyanate or guanidinium isothiocyanate, followed by, for example, phenol-chloroform extraction, silica-based column techniques (e.g. RNeasy Kit by Qiagen) and magnetic beads-based techniques (e.g. Dynabeads by Invitrogen). A preferred method involves guanidinium thiocyanate-extraction such as, e.g. TRIzol® Kit by Invitrogen.

Several methods are known to generate total RNA that is depleted from ribosomal RNA (rRNA). Depletion of the highly abundant rRNA fraction (80-90%) is desirable, for example, prior to performing an RNA-seq reaction, so that sequencing can be directed to sequencing of messenger RNAs, also termed “transcriptome”. Conventional methods for eliminating rRNA from total RNA samples include enrichment of polyadenylated (poly(A)) transcripts, and targeted depletion of rRNA. Targeted depletion is usually achieved by either rRNA pull-out using biotinylated sequence-specific probes (e.g., Illumina's Ribo-Zero and Thermo Fisher's RiboMinus) or RNase H-mediated degradation (e.g., NEB's NEBNext).

The step of isolating RNA from a sample obtained from an individual may be replaced by the provision of an RNA sample from an individual, thereby providing a method of typing a sample comprising gene expression products from breast cancer cells of an individual who is diagnosed with breast cancer, comprising (i) determining an expression level of at least 16 marker genes in the RNA sample to thereby provide an expression profile of said marker genes, wherein the marker genes are selected from the genes listed in Table 1; and (ii) comparing the individual's expression profile to a reference expression profile of the at least 16 marker genes; thereby typing the sample for a response following auxiliary immune therapy.

### 4.3. Marker Genes

The invention provides a set of at least 16 marker genes whose expression is correlated with a response, here pCR, to auxiliary immune therapy in breast cancer patients. Said at least 16 marker genes are selected from the list of genes provided in Table 1. Since not all breast cancer patients may benefit from auxiliary immune therapy, predicting a response before the actual start of the treatment may be part of an approach for optimal treating said individual. Said prediction may help a physician in selecting a treatment strategy for said individual. Preferably, a set of at least 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 marker genes from the marker genes listed in Table 1 is used, such as all 53 genes listed in Table 1. The marker genes are provided in Table 1 with gene name/symbol, prove sequence, correlation to pCR or RD, Ensembl ID, Human Genome Nomenclature Committee (HGNC) ID and a short description. In case of a discrepancy in the identification of a marker gene, the sequence of the probe is dominant. A positive correlation to pCR (indicated as “pCR” in column “Upregulated in” in Table 1) means that the level of expression of a gene is increased in a patient that positively responded to auxiliary immune therapy, when compared to a control. A positive correlation to RD means that the level of expression of a gene is increased in a patient that did not responded to auxiliary immune therapy, when compared to a control.

TABLE 1
Overview of gene markers to predict pathologic complete response (pCR) following
auxiliary immune therapy (SEQ ID Nos: 1-53, respectively top to bottom).

| | | | | | | |
|---|---|---|---|---|---|---|
| LAMP3 | ACCATGTTGACTTTCCTCATGTGTTTCCTT | pCR | 14582 | ENSG00000078081 | lysosomal-associated | 0.01056 |
| | ATGACTCAGTAAGTTGGCAAGGTCCTGACT | | | | membrane protein 3 | |
| IRF1 | CTTGGGTCATTGACCTCTCAGGGCCTGGCA | pCR | 6116 | ENSG00000125347 | interferon regulatory | 0.00533 |
| | GGCCAGTGTCTGGGTTTTTCTTGTGGTGTA | | | | factor 1 | |
| GZMB | AACCATGAAACGCTACTAACTACAGGAAGC | pCR | 4709 | ENSG00000100453 | granzyme B (cytotoxic T- | 0.02147 |
| | AAACTAAGCCCCCGCTGTAATGAAACACCT | | | | lymphocyte-associated | |
| | | | | | serine esterase 1) | |
| ICOS | AAGCTGTGCCTCGACACATCCTCATCCCAA | pCR | 5351 | ENSG00000163600 | inducible T-cell co- | 0.07668 |
| | GCATGGGACACCTCAAGATGAATAATAATT | | | | stimulator | |
| CCL19 | ACATCGTGAGGAACTTCCACTACCTTCTCA | pCR | 10617 | ENSG00000172724 | chemokine (C-C motif) | 0.04122 |
| | TCAAGGATGGCTGCAGGGTGCCTGCTGTAG | | | | ligand 19 | |
| IFNG | ACTTGAATGTCCAACGCAAAGCAATACATG | pCR | 5438 | ENSG00000111537 | interferon gamma | 0.07731 |
| | AACTCATCCAAGTGATGGCTGAACTGTCGC | | | | | |
| LCK | AGAAGCCATTAACTACGGGACATTCACCAT | pCR | 6524 | ENSG00000182866 | lymphocyte-specific | 0.02234 |
| | CAAGTCAGATGTGTGGTCTTTTGGGATCCT | | | | protein tyrosine kinase | |
| GBP1 | CAAAGATGCATTTACCTCTGTATCAACTCA | pCR | 4182 | ENSG00000117228 | guanylate binding | 0.02255 |
| | GGAAATCTCATAAGCTGGTACCACTCAGGA | | | | protein 1 | |
| CXCL13 | CAAGAGGCAAAGGAATCCATGTAGTAGATA | pCR | 10639 | ENSG00000156234 | chemokine (C-X-C motif) | 0.05888 |
| | TCCTCTGCTTAAAAACTCACTACGGAGGAG | | | | ligand 13 | |
| FAM30A | CCATTTTAAAGATGGCTACTTAGGACCATA | pCR | 19955 | ENSG00000123104 | KIAA0125 | 0.09835 |
| | TGGATGTTGTACTGATGTCATTTGACCACG | | | | | |
| CCL21 | CTTCCTTGTAAATATGATTTATACCTAACT | pCR | 10620 | ENSG00000137077 | chemokine (C-C motif) | 0.12293 |
| | GAATAAAAAGCTGTTCTGTCTTCCCACCCA | | | | ligand 21 | |
| IL2RG | CTTTCCTGTTTGCATTGGAAGCCGTGGTTA | pCR | 6010 | ENSG00000147168 | interleukin 2 receptor | 0.06579 |
| | TCTCTGTTGGCTCCATGGGATTGATTATCA | | | | gamma | |
| PDCD1 | GCTGGGACAAGGGATCCCCCTTCCCTGTGG | pCR | 8760 | ENSG00000188389 | programmed cell death 1 | 0.03469 |
| | TTCTATTATATTATAATTATAATTAAATAT | | | | | |
| PIM2 | GGTGAGGGGACCCTACTTTGTTATCCCAAG | pCR | 8987 | ENSG00000102096 | pim-2 oncogene | 0.03905 |
| | TGCTCTTATTCTGGTGAGAAGAACCTTAAT | | | | | |
| CXCL10 | GTCAAGCCATAATTGTTCTTAGTTTGCAGT | pCR | 10637 | ENSG00000169245 | chemokine (C-X-C motif) | 0.03494 |
| | TACACTAAAAGGTGACCAATGATGGTCACC | | | | ligand 10 | |
| GNLY | GTGTGTAGGACGGGGAGGTCACGATGGCGC | pCR | 4414 | ENSG00000115523 | granulysin | 0.01775 |
| | GACGTCTGCAGAAATTTCATGAGGAGGTAT | | | | | |
| CD19 | TACATGCCAGTGACACTTCCAGTCCCCTTT | pCR | 1633 | ENSG00000177455 | CD19 molecule | 0.04382 |
| | GTATTCCTTAAATAAACTCAATGAGCTCTT | | | | | |
| PTPN7 | TCACTCAGGACTTCTCTCCTGAAGAACACG | pCR | 9659 | ENSG00000143851 | protein tyrosine | 0.00742 |
| | CAGTGCTAAAACTGAGGATGATTTCCCTAA | | | | phosphatase, non- | |
| | | | | | receptor type 7 | |
| CXCR6 | TCAGAAGGCTCTTCTGACTACTGGGCAAAG | pCR | 16647 | ENSG00000172215 | chemokine (C-X-C motif) | 0.04855 |
| | AGTGTAGATCAGAGCAGCAGTGAAAACAAT | | | | receptor 6 | |
| TAP1 | TCCAGGATGAGTTACTTGAAATTTGCCTTG | pCR | 43 | ENSG00000168394 | transporter 1, ATP | 0.02032 |
| | AGTGTGTTACCTCCTTTCCAAGCTCCTCGT | | | | binding cassette | |
| | | | | | subfamily B member | |
| CD38 | TGAAAAATCCTGAGGATTCATCTTGCACAT | pCR | 1667 | ENSG00000004468 | CD38 molecule | 0.01067 |
| | CTGAGATCTGAGCCAGTCGCTGTGGTTGTT | | | | | |
| PDCD1LG2 | TGGAATACTCACGTGAGGGAACTTACTTTG | pCR | 18731 | ENSG00000197646 | programmed cell death | 0.05204 |
| | GCCAGCATTGACCTTCAAAGTCAGATGGAA | | | | 1 ligand 2 | |
| CCL13 | TGGCTATTATTTGACTTGTTGCTGGTTTGG | pCR | 10611 | ENSG00000181374 | chemokine (C-C motif) | 0.01151 |
| | GAGTTTATTTGAGTATTGCTGATCTTTTTC | | | | ligand 13 | |
| TIGIT | TGTGAAGCAAGGTTGTGTCTTGCGCATGGT | pCR | 26838 | ENSG00000181847 | T cell immunoreceptor | 0.01761 |
| | TAAAAATAAAGCATTGTCCTGCTTCCTAAG | | | | with Ig and ITIM | |
| | | | | | domains | |
| STAT1 | TTGAACCCTACACGAAGAAAGAACTTTCTG | pCR | 11362 | ENSG00000115415 | signal transducer and | 0.01905 |
| | CTGTTACTTTCCCTGACATCATTCGCAATT | | | | activator of | |
| | | | | | transcription 1 | |
| POU2AF1 | TTTTCTGGGAAATGACTTTTCTGGGAAATG | pCR | 9211 | ENSG00000110777 | POU class 2 associating | 0.06024 |
| | ACAGTTTCTTTGACATATTTTCTTTGCCCA | | | | factor 1 | |
| CD274 | AGAGTTTCCTTTCCCTCTTGGCCATATTCT | pCR | 17635 | ENSG00000120217 | programmed cell death 1 | 0.07446 |
| | GGTGTCAATGACAAGGAGTACCTTGGCTTT | | | | ligand 1 Precursor | |
| HSD11B1 | TGGGACTGTTCTGTCTCATGTTTATCTGAG | pCR | 5208 | ENSG00000117594 | hydroxysteroid (11-beta) | 0.03925 |
| | CTCTTATCTATGAAGACATCTTCCCAGAGT | | | | dehydrogenase 1 | |
| CD209 | AGGGCGGCCCTGTACTTTTTGTGGCTTGGT | pCR | 1641 | ENSG00000090659 | CD209 molecule | 0.07738 |
| | TTCTCTGGAAATGTCACCTTTTCGGGCGCA | | | | | |
| TIFAB | AACAATTACATTTGTGGTAGCTTGGGGTTA | pCR | 5963 | ENSG00000255833 | TRAF-interacting protein | 0.00324 |
| | CCTTCAGCTTTCAAATACCCACAGGCACGC | | | | with forkhead-associated | |
| | | | | | domain, family member B | |
| HLA-J | CCGCTACTACAACCAGAGCGAGGCGGGGTA | RD | 4967 | ENSG00000204622 | major histocompatibility | 0.01601 |
| | TCACATCCTCCAGGGAATGTTTGGCTGCGA | | | | complex, class I, J | |
| EDIL3 | ACATGACTGCCTATCAGTAGATTGATCTGT | RD | 30248 | ENSG00000164176 | EGF-like repeats and | 0.04046 |
| | ATTTAATATTCGTTAATTAAATCTGCAGTT | | | | discoidin 1-like domains 3 | |
| SMIM3 | AGAAGATAGCACAGATATCGGGATATTATT | RD | 20301 | ENSG00000256235 | chromosome 5 open | 0.00283 |
| | GTGTGAAAATGCTGCTTTTACTTTGATGTG | | | | reading frame 62 | |
| SLC22A15 | AGGCTAGGCTGGCCATCAGTTGCTTATTTC | pCR | 19010 | ENSG00000163393 | solute carrier family 22, | 0.01511 |
| | AGATGTGTCACTAAATTTTCCTTCTAGATG | | | | member 15 | |
| GPR114 | AGGCTCTGAGACTTTACTGGCCTATGCCTG | pCR | 21073 | ENSG00000159618 | G protein-coupled receptor | 0.00227 |
| (ADGRG5) | AGGCCTCTTTTCCTTTAACTCCCTAAATTA | | | | 114 | |
| TXNDC5 | ATACGCAAGGGGATGTGGATACTTGGCCCA | RD | 14150 | ENSG00000259040 | thioredoxin domain | 0.00303 |
| | AAGTAACTGGTGGTAGGAATCTTAGAAACA | | | | containing 5 | |
| FBXL16 | ATGCGGAAGATATAACCTTATATTTGGTAA | pCR | 2879 | ENSG00000127585 | F-box and leucine-rich | 0.14335 |
| | GTGTTTCTTGTGCTATTTTATCACGTGACC | | | | repeat protein 16 | |
| SEPTIN1 | ATTTCCTGAACCTGCGACGGATGCTGGTGC | pCR | 11190 | ENSG00000180096 | septin 1 | 0.01778 |
| | AGACACACCTGCAGGACCTGAAAGAGGTGA | | | | | |
| SOX10 | CATCGGAGGCCTTACCACTCCTATGACTCC | RD | 29944 | ENSG00000100146 | SRY (sex determining | 0.21869 |
| | TGTTTTCTCTCTCACAGATAGTGAGGGTCT | | | | region Y)-box 10 | |
| TENM3 | CCGAATGACCTTAAAGGTGATCGGCTTTAA | pCR | 34024 | ENSG00000218336 | teneurin transmembrane | 0.0085 |
| | CGAATATGTTTACATATGCATAGCGCTGCA | | | | protein 3 | |
| CNIH3 | CTCCCATCTGAAACCTGTGACTCAGGTTTA | RD | 26802 | ENSG00000143786 | cornichon homolog 3 | 0.01819 |
| | TGAATGGTGTTTGTGTAGCAACACATTGTG | | | | | |
| CA12 | CTGTCAAAAGACTCTGTATATCTTTTGTGG | RD | 1371 | ENSG00000074410 | carbonic anhydrase XII | 0.01112 |
| | ATGAGTTTTGTGAGAGAACAGAGAGACCAT | | | | | |
| IL21R | GGAAATTGGCGATGTCACCCGTGTACGGTA | pCR | 6006 | ENSG00000103522 | interleukin 21 receptor | 0.00452 |
| | CGCAGCCCAGAGCAGACCCTCAATAAACGT | | | | | |
| TNNT1 | GGAAGCCTGGGAGTGTTTGTCCCATCGGTA | RD | 11948 | ENSG00000105048 | troponin T type 1 | 0.02983 |
| | GCTTGAAATAAACGCTCCCCTCAGACACCC | | | | (skeletal, slow) | |
| KIF3A | GGGGAACAGTTCATACTATAGAGAGTTACA | RD | 6319 | ENSG00000131437 | kinesin family member 3A | 0.00911 |
| | GTTTAGATGTATGTGTAAATCGATTAGCTA | | | | | |
| RADIL | TAGGTTCTATTTATATGGCAGATGACGTGA | RD | 22226 | ENSG00000157927 | Ras association and D1L | 0.00099 |
| | AATTGTGATGTTTGTTACAGAGCTTTTATG | | | | domains | |
| GPRC5C | TCCAGCCAAATAGTGTTCTCGGGGTGGTGG | RD | 13309 | ENSG00000170412 | G protein-coupled | 0.00939 |
| | CTGGGCAGCGCCTATGTTTCTCTGGAGATT | | | | receptor, family C, | |
| | | | | | group 5, member C | |
| CRABP1 | TCCCCTGAGGAATATGTCATAGTTCTGAGC | pCR | 2338 | ENSG00000166426 | cellular retinoic acid | 0.06534 |
| | TGCCAGTGGACCGCCCTTTTCCCCTACCAA | | | | binding protein 1 | |
| FCRL5 | TGGCATATGGACTGAAAGAAACTATGCTAT | pCR | 18508 | ENSG00000143297 | Fc receptor-like 5 | 0.03233 |
| | TGGATCTCCTGGATCTCCAGCTTGCTGACT | | | | | |
| MCOLN2 | TGGGAAAGAGGACTCACAGTTAGCCAGCTG | pCR | 13357 | ENSG00000153898 | mucolipin 2 | 0.00131 |
| | ACCATGACTGAAGTTCCAGCTTTACTTTTT | | | | | |
| ITGAL | TGTCAGGGCGTGGGACATCTAGTAGGTGCT | pCR | 6148 | ENSG00000005844 | integrin alpha L | 0.0985 |
| | TGACATAATTTCACTGAATTAATGACAGAG | | | | | |
| CAPN13 | TTTGCAGAATAATTACACTATCTTATGTCT | RD | 16663 | ENSG00000162949 | calpain 13 | 0.06959 |
| | GGATCCTGATGATTTCACAGCTAAATGGCA | | | | | |
| IRF4 | TTTTGTATTGATTTTCACAGCTTTGAGGAA | pCR | 6119 | ENSG00000137265 | interferon regulatory | 0.01802 |
| | CATGCATAAGAAATGTAGCTGAAGTAGAGG | | | | factor 4 | |

### 4.4 Determining Expression Levels of Marker Genes

The determination of an expression level of one or more marker genes can be accomplished by any means known in the art such as Northern blotting, quantitative PCR (qPCR), microarray analysis or RNA-seq. Preferably, the expression levels of multiple marker genes are assessed simultaneously, by multiplex qPCR, microarray analysis or RNA-seq.

Microarray analysis involves the use of selected probes that are immobilized on a solid surface, an array. Said probes are able to hybridize to gene expression products such as mRNA, or derivates thereof such as cDNA. The probes are exposed to labeled sample gene expression products, or labelled derivates thereof, hybridized, washed, where after the abundance of gene expression products or derivates thereof in the sample that are complementary to a probe is determined by determining the amount of label that remains associated to a probe. The probes on a microarray may comprise DNA sequences, RNA sequences, or copolymer sequences of DNA and RNA. The probes may also comprise DNA and/or RNA analogues such as, for example, nucleotide analogues or peptide nucleic acid molecules (PNA), or combinations thereof. The sequences of the probes may be full or partial fragments of genomic DNA. The sequences may also be in vitro synthesized nucleotide sequences, such as synthetic oligonucleotide sequences.

In the context of the invention, a probe preferably is specific for a gene expression product of a gene as listed in Table 1. A probe is specific when it comprises a continuous stretch of nucleotides that are completely complementary, over the whole length, to a nucleotide sequence of a gene expression product, or a cDNA product thereof. A probe can also be specific when it comprises a continuous stretch of nucleotides that are partially complementary to a nucleotide sequence of a gene expression product of said gene, or a cDNA product thereof. Partially means that a maximum of 5 nucleotides, more preferable 4 nucleotides, more preferable 3 nucleotides, more preferable 2 nucleotides and most preferable one nucleotide differs from the corresponding nucleotide sequence of a gene expression product of said gene. The term complementary is known in the art and refers to a sequence that is related by base-pairing rules to the sequence that is to be detected. It is preferred that the sequence of the probe is carefully designed to minimize nonspecific hybridization to said probe.

The specificity of probe is further determined by the hybridization and/or washing conditions. The hybridization and/or washing conditions are preferably stringent, which are determined by inter alia the temperature and salt concentration of the hybridization and washing conditions, as is known to a person skilled in the art. An increased stringency will substantially reduce non-specific hybridization to a probe, while specific hybridization is not substantially reduced. Stringent conditions include, for example, washing steps for five minutes at room temperature 0.1× sodium chloride-sodium citrate buffer (SSC)/0.005% Triton X-102. More stringent conditions include washing steps at elevated temperatures, such as 37° Celsius, 45° Celsius, or 65° Celsius, either or not combined with a reduction in ionic strength of the buffer to 0.05×SSC or even 0.01×SSC, as is known to a skilled person.

It is preferred that the probe is, or mimics, a single stranded nucleic acid molecule. The length of a probe can vary between 15 bases and several kilo bases, and is preferably between 20 bases and 1 kilobase, more preferred between 40 and 100 bases, and most preferred about 60 nucleotides. A most preferred probe comprises about 60 nucleotides. Said probe is preferably identical over the whole length to a nucleotide sequence of a gene expression product of a gene, or a cDNA product thereof. In a method of the invention, probes comprising probe sequences as indicated in Table 1 can be employed.

To determine an RNA expression level by micro arraying, gene expression products in the sample are preferably labeled, either directly or indirectly, and contacted with probes on the array under conditions that favor duplex formation between a probe and a complementary molecule in the labeled gene expression product sample. The amount of label that remains associated with a probe after washing of the microarray can be determined and is used as a measure for the gene expression level of a nucleic acid molecule that is complementary to said probe.

Image acquisition and data analysis can subsequently be performed to produce an image of the surface of the hybridized array. For this, the array may be dried and placed into a laser scanner to determine the amount of labeled sample that is bound to a probe at a predetermined spot. Laser excitation will yield an emission with characteristic spectra that is indicative of the labelled sample that is hybridized to a probe molecule.

An array preferably comprises multiple spots encompassing a specific probe. A probe preferably is present in duplicate, in triplicate, in quadruplicate, in quintuplicate, in sextuplicate or in octuplicate on an array. The multiple spots preferably are at randomized opposition on an array to minimize bias. The amount of label that remains associated with the probe at each spot may be averaged, where after the averaged level can be used as a measure for the gene expression level of a nucleic acid molecule that is complementary to said probe. In addition, a gene product may be hybridized to two or more different probes that are specific for that gene product.

The determined RNA expression level can be normalized for differences in the total amounts of nucleic acid expression products between two separate samples by comparing the level of expression of one or more genes that are presumed not to differ in expression level between samples such as glyceraldehyde-3-phosphate-dehydro-genase, β-actin, and ubiquitin. Conventional methods for normalization of array data include global analysis, which is based on the assumption that the majority of genetic markers on an array are not differentially expressed between samples (Yang et al., 2002. Nucl Acids Res 30:15). Alternatively, the array may comprise specific probes that are used for normalization. These probes preferably detect RNA products from housekeeping genes such as glyceraldehyde-3-phosphate dehydrogenase and 18S rRNA levels, of which the RNA level is thought to be constant in a given cell and independent from the developmental stage or prognosis of said cell.

Another preferred method for determining RNA expression levels is by sequencing, preferably next-generation sequencing (NGS), of RNA samples, with or without prior amplification of the RNA expression products.

High throughput sequencing techniques for sequencing RNA, or RNA-seq, have been developed. NGS platforms, including Illumina® sequencing; Roche 454 Pyrosequencing®, ion torrent and ion proton sequencing, and ABI SOLID® sequencing, allow sequencing of fragments of DNA in parallel. Bioinformatics analyses are used to piece these fragments together by mapping the individual reads. Each base is sequenced multiple times, providing high depth to deliver accurate data and an insight into unexpected DNA variation. NGS can be used to sequence a complete exome including all genes or alternatively to sequence a number of individual genes.

Pyrosequencing detects the release of inorganic pyrophosphate (PPi) as particular nucleotides are incorporated into the nascent strand (Ronaghi et al., 1996. Analytical Biochemistry 242:84-9; Ronaghi, 2001. Genome Res 11:3-11; Ronaghi et al., 1998. Science 281:363; U.S. Pat. Nos. 6,210,891; 6,258,568; and 6,274,320, which are all incorporated herein by reference. In pyrosequencing, released PPi can be detected by being immediately conversion to adenosine triphosphate (ATP) by ATP sulfurylase, and the level of ATP generated is detected via luciferase-produced photons.

NGS also includes so called third generation sequencing platforms, for example nanopore sequencing on an Oxford Nanopore Technologies platform, and single-molecule real-time sequencing (SMRT sequencing) on a PacBio platform, with or without prior amplification of the RNA expression products.

Further high throughput sequencing techniques include, for example, sequencing-by-synthesis. Sequencing-by-synthesis or cycle sequencing can be accomplished by stepwise addition of nucleotides containing, for example, a cleavable or photobleachable dye label as described, for example, in U.S. Pat. Nos. 7,427,673; 7,414,116; WO 04/018497; WO 91/06678; WO 07/123744; and U.S. Pat. No. 7,057,026, all of which are incorporated herein by reference.

Sequencing techniques also include sequencing by ligation techniques. Such techniques use DNA ligase to incorporate oligonucleotides and identify the incorporation of such oligonucleotides and are inter alia described in U.S. Pat. Nos. 6,969,488; 6,172,218; and 6,306,597. Other sequencing techniques include, for example, fluorescent in situ sequencing (FISSEQ), and Massively Parallel Signature Sequencing (MPSS).

Sequencing techniques can be performed by directly sequencing RNA, or by sequencing a RNA-to-cDNA converted nucleic acid library. Most protocols for sequencing RNA samples employ a sample preparation method that converts the RNA in the sample into a double-stranded cDNA format prior to sequencing. Conversion of RNA into cDNA and/or cRNA using a reverse-transcriptase enzyme such as M-MLV reverse-transcriptase from Moloney murine leukemia virus, or AMV reverse-transcriptase from avian myeloblastosis virus, is known to a person skilled in the art.

Quantitative PCR (qPCR), or real-time PCR (RT-PCR), is a technique which is used to amplify and simultaneously quantify a template nucleic acid molecule such as an RNA. The detection of the amplification product can in principle be accomplished by any suitable method known in the art. The amplified products may be directly stained or labelled with radioactive labels, antibodies, luminescent dyes, fluorescent dyes, or enzyme reagents. Direct DNA stains include for example intercalating dyes such as acridine orange, ethidium bromide, ethidium monoazide or Hoechst dyes. These intercalating dyes are non-specific and bind to all double stranded DNA in the PCR. An increase in DNA products during amplification, results in an increased fluorescence intensity being measured. Another direct DNA detection method includes the use of sequence specific DNA probes consisting of a fluorescent reporter and quencher. Upon binding of the probe to its complementary sequence, polymerases of the PCR break the proximity of the reporter and the quencher, resulting in the emission of fluorescence. Commonly used reporter dyes include FAM (Applied Biosystems), HEX (Applied Biosystems), ROX (Applied Biosystems), YAK (ELITech Group) or VIC (Life Technologies) and commonly used quenchers include TAMRA (Applied Biosystems), BHQ (Biosearch Technologies) and ZEN (Integrated DNA Technologies). Alternatively, the amplified product may be detected by incorporation of labelled dNTP bases into the synthesized DNA fragments. Detection labels which may be associated with nucleotide bases include, for example, fluorescein, cyanine dye and BrdUrd.

For the simultaneous detection of multiple nucleic acid gene expression products, a multiplex qPCR can be used. In multiplex qPCRs, two or more template nucleic acid molecules are amplified and quantified in the same reaction. A commonly used method of achieving the simultaneously detection of multiple targets, is by using probes with different fluorescent dyes to distinguish distinct nucleic acid targets.

It is preferred in methods of the invention that genes are selected for normalization of the raw data. Preferred genes are genes of which the RNA expression levels are largely constant between individual samples comprising breast cancer cells from one individual, and between samples comprising breast cancer cells from different individuals. It will be clear to a skilled artisan that the RNA levels of said set of normalization genes preferably allow normalization over the whole range of RNA levels. An example of such a set of normalization genes is provided in WO 2008/039071, which is hereby incorporated by reference.

Normalization methods that may be employed include, for example, mean correction, linear combination of factors, Bayesian methods and non-linear normalization methods such quantile normalization. Preferred methods include non-parametric regression methods such as locally estimated scatterplot smoothing (LOESS; Jacoby, 2000. Electoral Studies 19:577-613) and locally weighted scatterplot smoothing (LOWESS; Cleveland et al., 1988. J American Statistical Association 83:596-610).

### 4.5 Prediction of an Individual's Response Following Auxiliary Immune Therapy

The invention provides a method for typing a sample to predict an individual's response to auxiliary immune therapy. Typing of a sample can be performed in various ways. In one method, the difference or similarity between a sample's expression profile and a previously established reference expression profile is determined. The sample's expression profile is composed of the expression levels of a set of marker genes in said sample. The reference expression profile is composed of the average expression levels of the same set of marker genes in a sample from a reference group. The reference group may comprise a single individual. Preferably the reference group comprises the average expression levels of at least 10, 25, 50, 100, 200 or 300 individuals. The reference group may include individuals with different responses to auxiliary immune therapy. The reference group may also include individuals that all show a response following auxiliary immune therapy (i.e. response reference group) or individuals not showing a response to auxiliary immune therapy (i.e. no response reference group). Alternatively, an expression profile of an individual can also be typed by comparing the individual's expression profile to multiple reference profiles. For example, the individual's expression profile can be compared to both reference profiles identified above (i.e. the response reference group and the no response reference group). If the expression profile of the individual's sample is substantially more similar to response reference group, when compared to the no response reference group, it will be predicted responsive.

The difference or similarity between an expression profile and one or more reference profiles can be determined by determining a correlation of the expression levels of marker genes in the profiles. For example, one can determine whether the expression levels of a subset of marker genes in a sample correlate to the expression levels of the same subset of marker genes in a reference profile. This correlation can be numerically expressed using a correlation coefficient. Several correlation coefficients can be used. Preferred methods are parametric methods which assume a normal distribution of the data. One of these methods is the Pearson product-moment correlation coefficient, which is obtained by dividing the covariance of the two variables by the product of their standard deviations.

Said correlations between the expression levels of marker genes in the individual's sample and the reference group, can be used to produce an overall similarity score for the set of marker genes used. A similarity score is a measure of the average correlation of gene expression levels of a set of genes in a sample from an individual with breast cancer and a reference profile. Said similarity score can, but does not need to be, a numerical value between +1, indicative of a high correlation between the gene expression level of the set of genes in a sample of said individual and said reference profile, and −1, which is indicative of an inverse correlation. A threshold can be used to differentiate between samples having a response, and samples having no response. Said threshold is an arbitrary value that allows for discrimination between samples from individuals with no response, and samples of individuals with a response. If a similarity threshold value is employed, it is preferably set at a value at which an acceptable number of patients with response would score as false negatives, and an acceptable number of patients with no response would score as false positives.

Based on the predictions made by the methods of the invention, one can determine a course of treatment of the individual with breast cancer. For example if the individual's expression profile is not substantially different from the no response group, or alternatively substantially different from the response group, this indicates that the individual is predicted to not show response to auxiliary immune therapy. In that case, it is not recommended to provide auxiliary immune therapy.

Preferably, the response to auxiliary immune therapy to be predicted is pCR. Alternatively, other responses to auxiliary immune therapy could be assessed such as residual cancer burden (RCB), (3-year) event-free survival (EFS) and distant recurrence-free survival (DRFS).

A prediction of an individual's response following auxiliary immune therapy may be combined with other predictive or prognostic signatures, such as MAMMAPRINT® (U.S. Pat. Nos. 7,514,209 and 9,909,185, both of which are incorporated herein by reference), BLUEPRINT® (U.S. Pat. Nos. 9,175,351 and 10,072,301, both of which are incorporated herein by reference), OncotypeDX®, MapQuantDX™ ProSigna® and EndoPredict®, and/or with presence or absence of biomarkers such as Oestrogen Receptor (ER), Progesterone Receptor (PR) and Human Epidermal Growth factor Receptor 2 (HER2/ERBB2).

4.6 Methods of Treating an Individual with Breast Cancer

A method of treatment of breast cancer is usually determined based on the grade of the cancer, the stage of the cancer, the cancer's molecular subtype, or any combination thereof. The most common breast cancer molecular subtypes include breast cancers expressing a molecular target such as ER, progesterone receptor (PR) or HER2, and are classified as ER positive, HER2 positive, or triple negative, a breast cancer that lacks the expression of three molecular targets. As an alternative, or in addition, said classification may be based on the Luminal-type, HER2-type and Basal-type (Perou et al., 2000. Nature 406:747-752; Krijgsman et al., 2012. Br Can Res Treatm 133:37-47), for example by using the BluePrint signature as described in U.S. Pat. Nos. 9,175,351 and 10,072,301, both of which are incorporated herein by reference.

For a non-metastatic breast cancer, primary treatment involves local treatment including surgery and often adjuvant post-operative radiotherapy. Surgery aims at the complete removal of the cancer tissue. In some instances, one or more of the axillary lymph nodes is removed as well.

Treatment of a nonmetastatic breast cancer may also involve systemic treatment depending on the molecular subtype of the breast cancer and is administered in addition to surgery. For hormone receptor positive (HR-positive; meaning ER and PR positive) breast cancer systemic treatment comprises endocrine therapy with or without chemotherapy. For HER2-positive breast cancer systemic therapy comprises chemotherapy combined with HER2-targeting therapy, by for example HER2-directed antibodies. For triple negative breast cancer, adjuvant therapy is mainly limited to chemotherapy.

Neoadjuvant immune therapy, including treatment with immunotherapeutic agents (often in combination with a chemotherapeutic drug) before surgery, has been shown to improve the progression-free survival of breast cancer patients and is a promising new treatment tool (Schmid et al., 2018. N Engl J Med 379 (22): 2108-2121).

An auxiliary immune therapy according to the invention may be combined with chemotherapy and/or targeted therapy. Preferably said targeted therapy comprises treatment with a PARP inhibitor, with a PI3K/AKT/mTOR inhibitor, or with both a PARP inhibitor and a PI3K/AKT/mTOR inhibitor. Furthermore, in a method of treating with auxiliary immune therapy provided by the invention, said auxiliary immune therapy can be combined with chemotherapy and/or targeted therapy. Preferably said targeted therapy is treatment with a PARP inhibitor and/or PI3K/AKT/mTOR inhibitor. Furthermore, in the use of auxiliary immune therapy according to the invention, said auxiliary immune therapy can by combined with chemotherapy and/or targeted therapy. Preferably said targeted therapy is treatment with a PARP inhibitor and/or PI3K/AKT/mTOR inhibitor.

A PARP inhibitor preferably is selected from olaparib (3-aminobenzamide, 4-(3-(1-(cyclopropanecarbonyl) piperazine-4-carbonyl)-4-fluorobenzyl) phthalazin-1 (2H)-one; AZD-2281; AstraZeneca), rucaparib (6-fluoro-2-[4-(methylaminomethyl)phenyl]-3,10-diazatricyclo[6.4.1.04,13]trideca-1,4,6,8 (13)-tetraen-9-one; Clovis Oncology, Inc.); niraparib tosylate ((S)-2-(4-(piperidin-3-yl)phenyl)-2H-indazole-7-carboxamide hydrochloride; MK-4827; GSK); talazoparib (11S,12R)-7-fluoro-11-(4-fluorophenyl)-12-(2-methyl-1,2,4-triazol-3-yl)-2,3,10-triazatricyclo[7.3.1.05,13]trideca-1,5 (13),6,8-tetraen-4-one; BMN-673; Pfizer); veliparib (2-[(2R)-2-methylpyrrolidin-2-yl]-1H-benzimidazole-4-carboxamide dihydrochloride benzimidazole carboxamide; ABT-888; Abbvie); pamiparib (2R)-14-fluoro-2-methyl-6,9,10,19-tetrazapentacyclo[14.2.1.02,6.08,18.012,17]nonadeca-1 (18),8,12 (17),13,15-pentaen-11-one; BGB-290; BeiGene); CEP-8983, and CEP 9722, a small-molecule prodrug of CEP-8983, a 4-methoxy-carbazole inhibitor (CheckPoint Therapeutics); E7016 (Eisai), PJ34 (2-(dimethylamino)-N-(6-oxo-5H-phenanthridin-2-yl)acetamide;hydrochloride) and 3-aminobenzamide.

A preferred PARP inhibitor is selected from the group consisting of olaparib, rucaparib, niraparib, talazoparib, and pamiparib.

Said PARP inhibitor preferably is administered orally, as a tablet or as a capsule. Said PARP inhibitor preferably is administered once or twice per day for a period of 1-24 weeks, for example once or twice daily for a 12 weeks period. The preferred dosage of selected PARP inhibitors is 100-500 mg twice daily, preferably 300-400 mg twice daily for olaparib; 200-1000 mg twice daily, preferably 400-600 mg twice daily for rucaparib; 50-500 mg twice daily, preferably 100-300 mg twice daily for niraparib tosylate; 0.2-2 mg twice daily, preferably 0.5-1 mg twice daily for talazoparib; 100-600 mg twice daily, preferably 200-400 mg twice daily for veliparib; and 300-100 mg twice daily, preferably 40-60 mg twice daily for pamiparib. A person skilled in the art will understand that the dosage in a combination according to the invention, may be at the low range of the indicated dosages, or even below the indicated dosages.

A PI3K/AKT/mTOR inhibitor is an inhibitor of the phosphoinositide 3-kinase (PI3K)/protein kinase (AKT)/mammalian target of rapamycin (mTOR) signalling pathway. This pathway regulates survival, proliferation, differentiation, apoptosis and other processes of breast cancer cells, and performs a pivotal function in the occurrence and development. Abnormal activation of this pathway is the most common pathogenesis of breast cancer (Martini et al., 2014. Ann Med 46:372-383). A large number of targeted drugs that act on various proteins of PI3K/AKT/mTOR pathway have been developed, providing a valuable tool for targeted therapy of HER2-negative breast cancer.

A PI3K inhibitor is preferably selected from buparlisib, pictilisib, alpelisib, idelalisib, copanlisib, duvelisib, parsaclisib, zandelisib (ME-401), NVP-BBD130, dactolisib (BEZ-235), NVP-BEZ-235 and gedatolisib (PKI-587). An AKT inhibitor (AKTi) is preferably selected from miransertib, ARQ 751, MK-2206, perifosine (KRX-0401), ATP competitive inhibitors such as ipatasertib, uprosertib, capivasertib and afuresertib. An mTOR inhibitor is preferably selected from everolimus, temsirolimus and sirolimus.

The invention provides a method of treating an individual with breast cancer comprising typing the individual according to the invention and treating the individual that is typed as having a response to auxiliary immune therapy, with auxiliary immune therapy, and treating the individual that is typed as not having a response to auxiliary immune therapy with chemotherapy, without the addition of immunotherapeutic agents.

The invention provides a method of treating an individual with breast cancer comprising typing the individual according to the invention and treating the individual that is typed as having a response to auxiliary immune therapy, with a combination of chemotherapy and immune therapy, and treating the individual that is typed as not having a response to auxiliary immune therapy with chemotherapy, without the addition of immunotherapeutic agents.

Said auxiliary immune therapy may be provided as adjuvant therapy or as neoadjuvant therapy.

The invention provides a use of auxiliary immune therapy for the treatment of an individual with breast cancer that is typed according to the invention as having response to auxiliary immune therapy.

The invention provides a use of a combination of auxiliary immune therapy and chemotherapy for the treatment of an individual with breast cancer that is typed according to the invention as having response to auxiliary immune therapy.

The invention further provides a use of auxiliary immune therapy for the treatment of an individual with breast cancer that is typed according to the invention as having response to auxiliary immune therapy, wherein said treatment further comprises chemotherapy such as a platinum-based compound and/or a taxane.

The invention provides auxiliary immune therapy for use in the treatment of an individual with breast cancer that is typed according to the invention as having response to auxiliary immune therapy.

The invention provides a combination of auxiliary immune therapy and chemotherapy for use in the treatment of an individual with breast cancer that is typed according to the invention as having response to auxiliary immune therapy.

The invention further provides auxiliary immune therapy for use in the treatment of an individual with breast cancer that is typed according to the invention as having response to auxiliary immune therapy, wherein said treatment further comprises chemotherapy such as a platinum-based compound and/or a taxane.

The invention provides a use of auxiliary immune therapy in the preparation of a medicament for the treatment of an individual with breast cancer that is typed according to the invention as having response to auxiliary immune therapy.

The invention provides a use of a combination of auxiliary immune therapy and chemotherapy in the preparation of a medicament for the treatment of an individual with breast cancer that is typed according to the invention as having response to auxiliary immune therapy.

The invention provides a use of auxiliary immune therapy in the preparation of a medicament for the treatment of an individual with breast cancer that is typed according to the invention as having response to auxiliary immune therapy, wherein said medicament further comprises a chemotherapy such as a platinum-based compound and/or a taxane.

Chemotherapeutic agents used in the treatment of individuals with cancer can be selected from following non-limiting examples: alkylating compounds such as bendamustine (Mundipharma Pharmaceuticals), busulfan (Pierre Fabre), carmustine (Bristol-Myers Squibb), chlorambucil (Aspen), cyclophosphamide (Baxter), dacarbazine (Pfizer), estramustine (Pfizer), ifosfamide (Baxter), lomustine (Kyowa Kirin Pharma), melphalan (GlaxoSmithKline), nimustine (Sankyo), procarbazine (Leadiant Biosciences), streptozotocin (Keocyt), temozolomide (Merck & Co), thiotepa (Adienne), treosulfan (Lamepro) and trofosfamide (Baxter); anthracyclines such as daunorubicin (Medac), doxorubicin (Pfizer), epirubicin (Pfizer), idarubicin (Pfizer), mitoxantrone (Pfizer), pirarubicin (Sanofi), pixantrone (Servier) and valrubicin (Endo Pharmaceuticals); anti-tumor antibiotics (not anthracyclines) such as bleomycin (Inovio Pharmaceuticals), dactinomycin (Ovation Pharmaceutical) and mitomycin (UroGen Pharma); platinum compounds such as cisplatin (Bristol Myers Squibb), carboplatin (Bristol Myers Squibb), oxaliplatin (Pfizer) and satraplatin (Yakult Honsha), antimetabolites such as azacitidine (Pfizer), capecitabine (Roche), cytarabine (Pfizer), cladribine (Janssen Pharmaceutica), clofarabine (Sanofi), decitabine (Janssen Pharmaceutica), fludarabine (Bayer), (5-)fluorouracil (FivepHusion), 5-fluoro-2′-deoxyuridine (Sigma-Aldrich), gemcitabine (Eli Lilly and Company), (6-)mercaptopurin (Aspen), methotrexate (Aldeyra Therapeutics), nelarabine (Novartis), pemetrexed (Eli Lilly and Company), pentostatin (Pfizer) and (6-)tioguanine (Aspen); anti-mitotic cytostatics such as vinblastine (Teva), vincristine (Teva), vindesine (EG), vinflunine (Pierre Fabre) and vinorelbine (Pierre Fabre), taxanes such as cabazitaxel (Sanofi), docetaxel (Sanofi), paclitaxel (Celgene) and tesetaxel (Odonate Therapeutics); non-taxane microtubule inhibitors such as eribulin (Eisai), indibulin (Baxter), ixabepilone (R-PHARM), patupilone (Novartis) and sagopilone (Bayer HealthCare); topo-isomerase inhibitors such as camptothecin (RTI International), etoposide (Bristol-Myers Squibb), irinotecan (Pfizer), teniposide (Bristol-Myers Squibb), tretinoin (Roche) and topotecan (Novartis); and histone deacetylase inhibitors such as chidamide (Chipscreen Bioscience, HUYA Bioscience International), entinostat (Syndax), mocetinostat (Mirati therapeutics), tacedinaline (Pfizer), domatinostat (4SC), romidepsin (Celgene), abexinostat (Xynomic Pharmaceuticals), belinostat (Onxeo), nanatinostat (CHR-3996, Chroma Therapeutics, Viracta Therapeutics), givinostat (ITALFARMACO), MPT0E028 (3-(1-benzenesulfonyl-2,3-dihydro-1H-indol-5-yl)-N-hydroxy-acrylamide), panobinostat (Secura Bio Limited), pracinostat ((E)-3-[2-butyl-1-[2-(diethylamino)ethyl]benzimidazol-5-yl]-N-hydroxyprop-2-enamide), quisinostat (Janssen Pharmaceuticals, NewVac), resminostat (4SC, Yakult Honsha), ricolinostat (Regenacy Pharmaceutica), trichostatin A (Vanda Pharmaceuticals), vorinostat (suberanilohydroxamic acid or SAHA, Merck & Co), butyric acid, 4-phenylbutyric acid, pivanex (pivaloyloxymethyl butyrate), valproic acid (2-propylpentanoic acid), cambinol (5-[(2-Hydroxynaphthalen-1-yl)methyl]-6-phenyl-2-thioxo-2,3-dihydropyrimidin-4 (1H)-one), selistat (EX-527, AOP Orphan Pharmaceuticals AG), nicotinamide (pyridine-3-carboxamide) and sirtinol (2-[[(2-hydroxy-1-naphthalenyl)methylene]amino]-N-(1-phenylethyl)-benzamide).

Chemotherapy used in the treatment of individuals with breast cancer and typed according to the invention may comprises a therapeutically effective amount of any of the chemotherapeutic agents known to treat cancer patients. Said chemotherapeutic agent preferably includes a taxane, a platinum compound, an anthracycline or alkylating compound. Said taxane preferably is paclitaxel, docetaxel or cabazitaxel. Said taxane is preferably administered intravenously, preferably by infusion. Said taxane preferably is repeatedly administered, for example once every week, once every two weeks, or once every three weeks. For example, paclitaxel may be administered at a dosage of 75-200 mg/m², such as about 80 mg/m², every 1-4 weeks; docetaxel may be administered at a dosage of 40-100 mg/m², such as about 60 mg/m², every 1-4 weeks; and cabazitaxel may be administered at a dosage of 5-75 mg/m², such as about 20 mg/m², every 1-4 weeks. Said platinum compound is preferably administered intravenously, preferably by infusion. For example, carboplatin may be administered at a dosage of 100-600 mg/m², such as about 360 mg/m², every 1-4 weeks and cisplatin may be administered at a dosage of 10-120 mg/m², such as about 75 mg/m², every 1-4 weeks. Said anthracycline is preferably administered intravenously, preferably by infusion. Said anthracycline preferably is repeatedly administered, for example once every week, once every two weeks, or once every three weeks. Said anthracycline is preferably administered intravenously, preferably by infusion. For example, doxorubicin may be administered at a dosage of 20-400 mg/m², such as about 60-75 mg/m², every 1-4 weeks and epirubicin may be administered at a dosage of 20-140 mg/m², such as about 60-90 mg/m², every 1-4 weeks. Said alkylating compound is preferably cyclophosphamide. Said alkylating compound is preferably administered per oral or intravenously, preferably by infusion. Said alkylating compound preferably is repeatedly administered, for example once every week, once every two weeks, or once every three weeks. For example, cyclophosphamide may be administered at a dosage of 30-800 mg/m², such as about 600 mg/m², every 1-4 weeks. A person skilled in the art will understand that the dosage in a combination according to the invention, may be at the low range of the indicated dosages, or even below the indicated dosages.

Another preferred chemotherapy used in the treatment of individuals with breast cancer and typed according to the invention, comprises a combination of two or more chemotherapeutic agents. Examples of a preferred combination of chemotherapeutic agents include a combination of paclitaxel and carboplatin, a combination of paclitaxel and gemcitabine, a combination of doxorubicin and cyclophosphamide (often referred to as “AC”), a combination of doxorubicin, cyclophosphamide and paclitaxel (often referred to as “AC-P”), a combination of doxorubicin, cyclophosphamide and docetaxel (often referred to as “AC-T”), a combination of doxorubicin and docetaxel (often referred to as “AT”), a combination of cyclophosphamide, methotrexate and fluorouracil (often referred to as “CMF”), a combination of epirubicin, cyclophosphamide, methotrexate and fluorouracil (often referred to as “E-CMF”), a combination of epirubicin and cyclophosphamide (often referred to as “EC”), a combination of epirubicin, cyclophosphamide and paclitaxel (often referred to as “EC-P”), a combination of epirubicin, cyclophosphamide and docetaxel (often referred to as “EC-T”), a combination of fluorouracil, doxorubicin and cyclophosphamide (often referred to as “FAC” or “CAF”), a combination of fluorouracil, epirubicin and cyclophosphamide (often referred to as “FEC”), a combination of fluorouracil, epirubicin, cyclophosphamide and paclitaxel (often referred to as “FEC-P”), a combination of fluorouracil, epirubicin, cyclophosphamide and docetaxel (often referred to as “FEC-T”), a combination of docetaxel, doxorubicin and cyclophosphamide (often referred to as “TAC”) and a combination of docetaxel and cyclophosphamide (often referred to as “TC”). In said combination, chemotherapeutic agents are preferably administered intravenously, preferably by infusion. In said combination chemotherapeutic agents preferably are repeatedly administered, for example once every week, once every two weeks, or once every three weeks. In said combination chemotherapeutic agent have dosages preferably as follows: paclitaxel may be administered at a dosage of 75-200 mg/m², such as about 80 mg/m², every 1-4 weeks; carboplatin may be administered at a dosage of 100-300 mg/m², such as about 300 mg/m², every 1-4 weeks; carboplatin may be administered at a dosage of 100-300 mg/m², such as about 300 mg/m², every 1-4 weeks; gemcitabine may be administered at a dosage of 500-3500 mg/m², such as about 1250 mg/m², every 1-4 weeks; cyclophosphamide may be administered at a dosage of 30-800 mg/m², such as about 600 mg/m², every 1-4 weeks; methotrexate may be administered at a dosage of 10-100 mg/m², such as about 40 mg/m², every 1-4 weeks; doxorubicin may be administered at a dosage of 10-100 mg/m², such as about 50 mg/m², every 1-4 weeks; fluorouracil may be administered at a dosage of 100-4000 mg/m², such as about 500 mg/m², every 1-4 weeks; epirubicin may be administered at a dosage of 50-200 mg/m², such as about 100 mg/m². In said combination methotrexate and cyclophosphamide, can be administered per oral or intravenously.

Immunotherapeutic agents used in the treatment of individuals with breast cancer can be selected from cancer vaccines, adoptive cell therapy, cytokines, but the most common type of immune therapy involves immune checkpoint inhibitors. An immune checkpoint inhibitor is an inhibitor of CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328. Said inhibitor preferably is a PD1/PDL1 inhibitor and/or an inhibitor of CTLA-4. Suitable immune checkpoint inhibitors are CTLA-4 inhibitors such as antibodies, including ipilimumab (Bristol-Myers Squibb) and tremelimumab (MedImmune); PD1/PDL1 inhibitors such as antibodies, including pembrolizumab (Merck), sintilimab (Eli Lilly and Company), tislelizumab (BeiGene), toripalimab (Shangai Junshi Biosciense Company), spartalizumab (Novartis), camrelizumab (Jiangsu HengRui Medicine C), nivolumab and MDX-1105 (Bristol-Myers Squibb), pidilizumab (Medivation/Pfizer), MEDI0680 (AMP-514; AstraZeneca), cemiplimab (Regeneron) and PDR001 (Novartis); fusion proteins such as a PD-L2 Fc fusion protein (AMP-224; GlaxoSmithKline); atezolizumab (Roche/Genentech), avelumab (Merck/Serono and Pfizer), durvalumab (AstraZeneca), KN035 (Jiangsu Alphamab Biopharmaceuticals Company), Cosibelimab (CK-301; Checkpoint Therapeutics), BMS-936559 (Bristol-Myers Squibb), BMS-986189 (Bristol-Myers Squibb); and small molecule inhibitors such as PD-1/PD-L1 Inhibitor 1 (WO2015034820; (2S)-1-[[2,6-dimethoxy-4-[(2-methyl-3-phenylphenyl)methoxy]phenyl]methyl]piperidine-2-carboxylic acid), BMS202 (PD-1/PD-L1 Inhibitor 2; WO2015034820; N-[2-[[[2-methoxy-6-[(2-methyl [1,1′-biphenyl]-3-yl)methoxy]-3-pyridinyl]methyl]amino]ethyl]-acetamide), PD-1/PD-L1 Inhibitor 3 (WO/2014/151634; (3S,6S,12S,15S,18S,21S,24S,27S,30R,39S,42S,47aS)-3-((1H-imidazol-5-yl)methyl)-12,18-bis((1H-indol-3-yl)methyl)-N,42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4,7,10,13), CA-170 (Curis) and ladiratuzumab vedotin (Seattle Genetics).

The auxiliary immune therapy used in the treatment of individuals with breast cancer and typed according to the invention can comprises any of the immunotherapeutic agents known to treat cancer patients. Said immunotherapeutic agent preferably includes an immune checkpoint inhibitor. Said immune checkpoint inhibitor is preferably administered intravenously, preferably by infusion. Said immune checkpoint inhibitor preferably is administered once every 2-4 weeks for a period of 1-24 weeks. The preferred dosage of selected immune checkpoint inhibitors is 2-4 mg/kg. preferably about 3 mg/kg every 2-4 weeks, or 240-480 mg every 2-4 weeks for ipilimumab; 100-400 mg, preferably about 200 mg every 2-4 weeks, preferably every 3 weeks for pembrolizumab; 100-500 mg, preferably 240-480 mg every 2-4 weeks, preferably every 2 weeks for nivolumab; 2-12 mg/kg. preferably 4-8 mg/kg every 2-4 weeks, preferably every 4 weeks for pidilizumab; 100-500 mg, preferably about 350 mg every 2-4 weeks, preferably every 3 weeks for cemiplimab; 600-1800 mg, preferably about 1200 mg every 2-4 weeks, preferably every 3 weeks for atezolizumab; 400-1200 mg, preferably about 800 mg, every 2-4 weeks, preferably every 2 weeks for avelumab; and 5-15 mg/kg, preferably about 10 mg/kg, or 1000-2000 mg, preferably about 1500 mg, every 2-4 weeks, preferably every 2 weeks for durvalumab. A person skilled in the art will understand that the dosage in a combination according to the invention, may be at the low range of the indicated dosages, or even below the indicated dosages.

In this invention, preferably, the auxiliary immune therapy includes a monoclonal antibody targeting programmed cell death protein 1 (PD-1) receptors of lymphocytes such as pembrolizumab.

For the purpose of clarity and a concise description, features are described herein as part of the same or separate aspects and preferred embodiments thereof, however, it will be appreciated that the scope of the invention may include embodiments having combinations of all or some of the features described.

The invention will now be illustrated by the following examples, which are provided by way of illustration and not of limitation and it will be understood that many variations in the methods described and the amounts indicated can be made without departing from the spirit of the invention and the scope of the appended claims.

### 5 EXAMPLES

### Example 1: Identification and Performance of 53 Marker Genes for the Prediction of pCR Following Neoadjuvant Immune Therapy

### Materials and Methods

### Sample Collection and Processing

Tumor samples were collected from 69 patients from the I-SPY 2 TRIAL (NCT01042379) that were treated with paclitaxel and pembrolizumab (here referred as “pembro” dataset, Table 2), all with a MammaPrint High Risk profile, and with either basal or luminal type by the BluePrint test. Pembrolizumab was administered at 200 mg intravenously once every 3 weeks (q3w) with Paclitaxel administered at 80 mg/m2 weekly (q1w) for 12 total weeks. This regimen was followed by a combination of doxorubicin (also known as Adriamycin) and cyclophosphamide (AC) chemotherapy (q2w or q3w) for 4 weeks. All the included patients have HER2-negative tumours. From these 69 patients, 31 patients had pCR, while 38 did not (Table 2). RNA was isolated from pre-treatment FF tissue (Qiagen RNeasy Mini Kit), and single channel full genome microarrays (Agilent) were processed between 2015 and 2016.

Furthermore, an independent data set comprising pre-treatment, FFPE samples of a total of 70 breast cancer patients from an I-SPY 2 durvalumab/olaparib arm, all with a MammaPrint High Risk profile, were analysed (referred as “durva” dataset, Table 2). All samples were HER2 negative. Sample collection and processing was performed similarly as for the “pembro” dataset.

A set of 73 samples (referred to “Quantum Leap” dataset, Table 2) for which a core biopsy was fresh frozen and another biopsy of the same tumour was formalin fixed and paraffin embedded, was used for translation of the signature developed in FF samples to be able to read out in FFPE tissue. From these tumour samples RNA was extracted (Qiagen RNeasy Mini Kit) from both FF and FFPE tissue and hybridized to the same type of microarrays (Agilent, full genome)

TABLE 2
Descriptions of the I-SPY 2 datasets (“pembro” and “durva”) and
Quantum Leap dataset. The distribution of patients that responded
to therapy per clinical subtype and the BluePrint subtypes are
indicated. All patients enrolled had MammaPrint High Risk profile.

| | N tumor | pCR | | RD | | BluePrint | |
|---|---|---|---|---|---|---|---|
| Dataset | samples | HR+ | HR− | HR+ | HR− | Basal | Luminal |
| Pembro | 69 FF | 12 | 19 | 28 | 10 | 38 | 31 |
| Durva | 70 FFPE | 20 | 9 | 29 | 12 | 38 | 32 |
| Quantum | 73 FF | NA | NA | NA | NA | NA | NA |
| Leap | 73 FFPE | NA | NA | NA | NA | NA | NA |

Abbreviations, pCR: pathological complete response; RD: residual disease; FF: Fresh Frozen; FFPE: formalin-fixed paraffin-embedded.

To evaluate the stability (precision) a control dataset of FFPE samples was used. Different pools (categories) of samples reflecting the different breast cancer subtypes were selected and RNA was extracted (Qiagen RNeasy Mini Kit). In total 4 different controls were available (Table 6) and hybridized on 3313 full genome microarrays (Agilent). The controls amounted to 372, 448, 723 and 503 data points respectively with a time span between October 2018 and June 2020, for PBCL2, PHTR2 and PLEP3, and between September 2019 and June 2020 for PHHE2.

### Prevalence Assessment Data

For the prevalence, both pembro and durva datasets and 1793 FFPE 44K samples from FLEX studies (NCT03053193) were used. The distribution of HR status in the FLEX samples is as follows: 267 HR− (TN) and 3568 HR+. The distribution of BluePrint subtype in the FLEX samples is as follows: 1523 Luminal and 370 Basal. No HER2-type samples were used.

### Analysis Methods

To identify the genes that are most predictive of immune sensitivity, gene selection was performed using the R package ‘matrixStats’ and base R comparing pCR and RD groups by iteratively splitting the dataset in training (n−1) and test (1), balancing them on hormonal receptor (HR) status. This process was repeated for 100 iterations for all 69 FF samples. Within each sample iteration of 100 comparisons, the average effect size (ES) was saved and candidate genes required an average ES>0.45 as significance cut-off criteria in all 69 sample iterations. Alongside the ES, also P-values were captured. Next to an ES cut-off, the candidate genes also required a minimum correlation of 0.5 between FF and FFPE expression.

The index of the created gene classifier ImPrint is calculated using the same classifier as for MammaPrint. In short, a correlation (Pearson correlation coefficient) is determined between the centroids of the pCR group and RD group, resulting in a correlation based prediction index. Next, this correlation based index is scaled between 0 and 100 using a sigmoid function (M Bakr and M Negm, 2012. Elsevier 174:223-260), wherein a decision threshold is set at 50 (i.e. samples scoring above 50 are predicted to achieve pCR when treated with immune checkpoint inhibitors (ICI), which assumes having sufficient biological evidence of immune sensitivity).

### Validation

Various methods were considered such as using leave one out cross validation generated and translated templates to perform an independent validation. Templates were generated to test performance in pembro FF samples, which were translated to a FFPE template through the matched Quantum Leap samples. By using the FF template on FF Quantum Leap samples and selecting those that scored within the 5th and 95th quantile, a template was created in FF Quantum Leap with the exact same samples. This template was thereafter used for prediction on durva FFPE samples. This validation was mainly meant to prove the signature was predictive in the most independent approach possible. Next, the FFPE performance was tested through generating FFPE templates in durva directly. The accuracy, sensitivity, specificity, and F1 score were also compared with other known gene signatures.

### Results

### Selected Genes

From the list of candidate genes, a selection was made of previously known immunological relevant genes (n=30) and those most predictive non-target genes (n=23) for which the number was decided through best predictive performance in the training data. This resulted in a signature of in total 53 unique genes. The genes are shown in Table 1 with the 95th percentile p-value determined over 69 iterations. All genes have significant P-values in most iterations, however, taking the 95th percentile gives a fair representation of the genes while almost all remain below the generally accepted p-value threshold of 0.05.

The predictive value was firstly assessed in the pembro dataset during the development of the signature. An overall accuracy of 88% indicated strong predictive power regarding immune sensitivity (Table 3). Next, the pembro FF template was translated through matched FF and FFPE samples to perform predictions on durva. An overall accuracy of 77% confirmed predictive value towards immune sensitivity (Table 4). The actual FFPE generated templates resulted in an higher overall accuracy of 81% in durva (Table 5).

TABLE 3
Performance of ImPrint in the pembro dataset with leave
on out cross-valitation (LOO-CV) and FF templates.

| | TP | FP | FN | TN | Sensitivity | Specificity | Accuracy | F1 | S/T |
|---|---|---|---|---|---|---|---|---|---|
| Pembro | 29 | 6 | 2 | 32 | 0.94 | 0.84 | 0.88 | 0.88 | 69/69 |
| (LOO-CV) | | | | | | | | | |
| All HR+ | 10 | 3 | 2 | 25 | 0.83 | 0.89 | 0.88 | 0.80 | 40/69 |
| Luminal HR+ | 4 | 1 | 2 | 24 | 0.67 | 0.96 | 0.90 | 0.73 | 31/40 |
| Basal HR+ | 6 | 2 | 0 | 1 | 1.00 | 0.33 | 0.78 | 0.86 | 9/40 |
| All TN (only basal) | 19 | 3 | 0 | 7 | 1.00 | 0.70 | 0.90 | 0.93 | 29/69 |

Abbreviations, TP: true positive; FP: false positive; FN: false negative; TN: true negative; S/T: samples out of total.

TABLE 4
Performance of ImPrint in the durva dataset with FF to FFPE translated templates.

| | TP | FP | FN | TN | Sensitivity | Specificity | Accuracy | F1 | S/T |
|---|---|---|---|---|---|---|---|---|---|
| Durva | 17 | 4 | 12 | 37 | 0.59 | 0.90 | 0.77 | 0.68 | 70/70 |
| (QL derived) | | | | | | | | | |
| All HR+ | 11 | 1 | 9 | 28 | 0.55 | 0.97 | 0.80 | 0.69 | 49/70 |
| All TN (only basal) | 6 | 3 | 3 | 9 | 0.67 | 0.75 | 0.71 | 0.67 | 21/70 |

Abbreviations, TP: true positive; FP: false positive; FN: false negative; TN: true negative; S/T: samples out of total.

TABLE 5
Performance of ImPrint in the durva dataset with leave
on out cross-valitation (LOO-CV) and FFPE templates.

| | TP | FP | FN | TN | Sensitivity | Specificity | Accuracy | F1 | S/T |
|---|---|---|---|---|---|---|---|---|---|
| Durva (LOO-CV) | 22 | 6 | 7 | 35 | 0.76 | 0.85 | 0.81 | 0.77 | 70/70 |
| All HR+ | 14 | 1 | 6 | 28 | 0.70 | 0.97 | 0.86 | 0.80 | 49/70 |
| Luminal HR+ | 0 | 1 | 4 | 27 | 0.00 | 0.96 | 0.84 | NaN | 32/49 |
| Basal HR+ | 14 | 0 | 2 | 1 | 0.88 | 1.00 | 0.88 | 0.93 | 17/49 |
| All TN (only basal) | 8 | 5 | 1 | 7 | 0.89 | 0.58 | 0.71 | 0.73 | 21/70 |

Abbreviations, TP: true positive; FP: false positive; FN: false negative; TN: true negative; S/T: samples out of total.

### Conclusion

A 53 gene classifier, called ImPrint, was found that can predict with 88% accuracy if a patient would achieve pCR or not upon paclitaxel and pembrolizumab treatment. Importantly, the classifier can be used on both FF and FFPE conserved tissues samples. In pooled control samples (Table 6), relative stability was between 98.42% and 99.00% (Table 6). The 53 genes of the final signature are shown in Table 1.

TABLE 6
Relative stability of control dataset.

| Name | Alias | Count | Mean | Median | SD | CV | RSD | Stability |
|---|---|---|---|---|---|---|---|---|
| PBCL2 | Control 1 | 372 | 32.898 | 32.831 | 1.070 | 6.760 | 1.070 | 98.930 |
| PHHE2 | Control 2 | 448 | 39.769 | 39.681 | 1.255 | 12.590 | 1.255 | 98.745 |
| PHTR2 | Control 3 | 723 | 44.191 | 44.086 | 1.580 | 27.422 | 1.580 | 98.420 |
| PLEP3 | Control 4 | 503 | 31.620 | 31.593 | 0.999 | 5.968 | 0.999 | 99.001 |

Abbreviations: SD: standard deviation; CV: coefficient of variation; RSD: relative standard deviation.

### Example 2: Determination of Minimal Gene Number

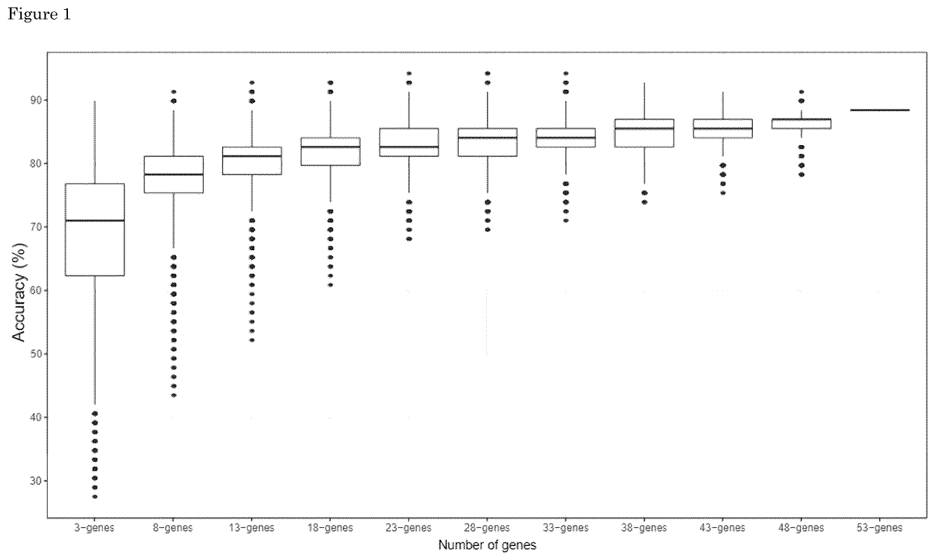

The performance of the gene signature ImPrint was evaluated for different minimal amounts of genes, starting with a minimal of 3 genes, increasing with intervals of 5 genes. For each amount, a total of up to 10 k different random combinations were evaluated, whenever possible. The results are provided in FIG. 1. From 10.000 iterations of 3 randomly selected genes over 75% had at least an accuracy of 60%, with some combinations even reaching up to 90%.

### Example 3: Comparison with Other Signatures

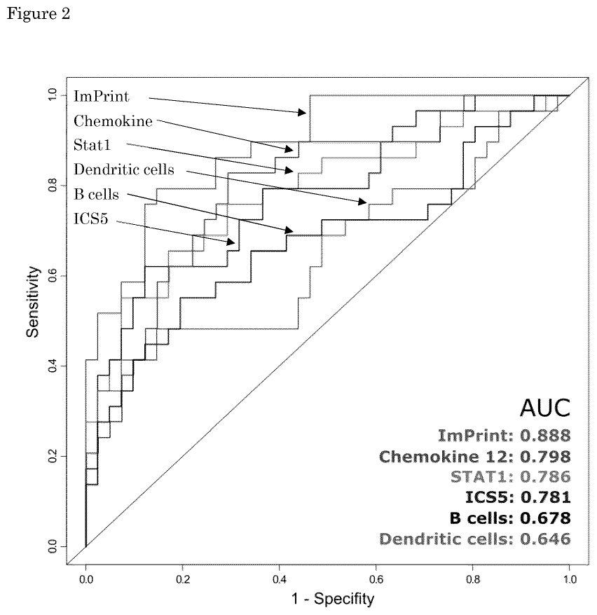

To assess the added value of ImPrint, predictions were compared to that of other published immune-related signatures that have been used in the Durvalumab/Olaparib validation dataset. As can be seen in FIG. 2 and Table 7, the ImPrint signature outperforms the signatures it was compared with.

TABLE 7
Multiple statistics on performance in the Durvalumab/Olaparib dataset
comparing ImPrint with other published signatures. Reference to
the following studies is made, Ref 1: Rody et al., 2009. Breast
Cancer Res 11: R15; Ref 2: Coppola et al., 2011. Am J Pathol 179:
37-45; Ref 3: Yau et al., 2013. Breast Cancer Res 15: R103; and
Ref 4: Danaher et al., 2017. J Immunother Cancer 5: 18.

| | | | | | F1 |
|---|---|---|---|---|---|
| | | Accuracy | Sensitivity | Specificity | score |
| ImPrint | | 0.81 | 0.76 | 0.85 | 0.77 |
| Chemokine 12 | Ref 1 | 0.74 | 0.72 | 0.76 | 0.70 |
| STAT1 | Ref 2 | 0.73 | 0.66 | 0.78 | 0.67 |
| ICS5 | Ref 3 | 0.67 | 0.72 | 0.63 | 0.65 |
| B Cells | Ref 4 | 0.66 | 0.59 | 0.71 | 0.59 |
| Dendritic cells | Ref 4 | 0.64 | 0.48 | 0.76 | 0.53 |

### Example 4: Prevalence of ImPrint

The prevalence of the ImPrint score was determined by dividing the number of positive outcomes over the total number of outcomes and these were reported for each subcategory (by HR status and BluePrint subtypes, Table 8).

TABLE 8
Prevalence for ImPrint in Pembrolizumab, Durvalumab/Olaparib,
and FLEX datasets, separately shown for BluePrint
and clinical subtype.

| | Immune- | Immune- | | |
|---|---|---|---|---|
| | sensitive | insensitive | Prevalence | Samples/Total |
| Pembro | 35 | 34 | 50.72% | 69/69 |
| All HR+ | 13 | 27 | 32.50% | 40/69 |
| Luminal HR+ | 5 | 26 | 16.13% | 31/40 |
| Basal HR+ | 8 | 1 | 88.89% | 9/40 |
| All TN (all basal) | 22 | 7 | 75.86% | 29/69 |
| Durva | 28 | 42 | 40.00% | 70/70 |
| All HR+ | 15 | 34 | 30.61% | 49/70 |
| Luminal HR+ | 1 | 31 | 3.13% | 32/49 |
| Basal HR+ | 14 | 3 | 82.35% | 17/49 |
| All TN (all basal) | 13 | 8 | 61.90% | 21/70 |
| FLEX | 320 | 1573 | 16.90% | 1793/1793 |
| All HR+ | 159 | 1479 | 9.71% | 1638/1793 |
| Luminal HR+ | 69 | 1436 | 4.58% | 1505/1638 |
| Basal HR+ | 90 | 43 | 67.67% | 133/1638 |
| All TN | 161 | 94 | 63.14% | 255/1793 |
| Luminal TN | 4 | 14 | 22.22% | 18/255 |
| Basal TN | 157 | 80 | 66.24% | 237/255 |

### Example 5: ImPrint Immune Signature in 10,000 Early-Stage Breast Cancer Patients from the Real-World FLEX Database

### Methods

FLEX (NCT03053193) is an ongoing registry trial with 97 sites open in the United States and 2 international sites. Patients enrolled in FLEX have early-stage breast cancer and receive standard of care MammaPrint (MP) testing with or without BluePrint (BP) molecular subtyping and consent to clinically annotated full genome data collection. MP is a 70-gene risk of distant recurrence signature that classifies patients as Low Risk or High Risk. MP High Risk can be further stratified into High 1 and High 2, which have demonstrated differences in chemosensitivity and pCR rates in the I-SPY2 TRIAL (NCT01042379). BP, an 80-gene molecular subtyping signature, categorizes patients' tumors as Luminal-, HER2- or Basal-Type.

### Results

Of the 10,021 patients, 9.1% of the FLEX patient population are ImPrint+ and are predicted to have a meaningful pCR rate with immune checkpoint inhibitors. Younger (≤50 years) or pre/peri-menopausal patients, patients with larger or node-positive tumors, and patients of Black or Latin race/ethnicity independently had a higher likelihood of having ImPrint+ tumors (Table 9). ImPrint+ tumors were identified in all clinical subtypes by IHC. There is a higher likelihood of ImPrint+ tumors being MP High 2 or BP Basal-Type tumors. Within BP Basal tumors, 74.7% of HR+ and 66.0% of HR− tumors were ImPrint+.

The focus of immune therapy trials has been on patients with HR-HER2-, MP High Risk patients. Indeed, most patients who are predicted to benefit have MP High 2 or BP Basal-Type tumors, including some HR+ patients, which is consistent with I-SPY2 results. Importantly, this large real-world dataset enables the identification of populations who may benefit from immune therapy outside of traditional clinical trial populations and supports the testing of checkpoint inhibitors in the immune-positive subtype. Younger women and patients of Black or Latin race/ethnicity who typically have more aggressive tumors also have higher proportions of ImPrint+ tumors. Thus, it is critical that these populations be included in clinical trials.

TABLE 9
Clinical characteristics of ImPrint+ and ImPrint− tumors.

| | | | |
|---|---|---|---|
| ImPrint+ | ImPrint− | Total | Significance |

Age

| | | | | | | | |
|---|---|---|---|---|---|---|---|
| >50 years | 586 | (7.9%) | 6814 | (92.1%) | 7400 | (80.1%) | p < 0.001 |
| ≤50 years | 265 | (14.4%) | 1572 | (85.6%) | 1837 | (19.9%) | |

Mensopausal status

| | | | | | | | |
|---|---|---|---|---|---|---|---|
| Pre-/peri- | 231 | (13.0%) | 1544 | (87.0%) | 1775 | (22.9%) | p < 0.001 |
| Post- | 490 | (8.2%) | 5500 | (91.8%) | 5990 | (77.1%) | |

Race/ethnicity

| | | | | | | | |
|---|---|---|---|---|---|---|---|
| Asian | 10 | (7.7%) | 120 | (92.3%) | 130 | (1.7%) | p < 0.001 |
| Black or African American | 141 | (18.9%) | 605 | (81.1%) | 746 | (9.9%) | |
| Latin American | 46 | (15.2%) | 256 | (84.8%) | 302 | (4.0%) | |
| White or Caucasian | 471 | (7.4%) | 5853 | (92.6%) | 6324 | (84.3%) | |

Tumor stage

| | | | | | | | |
|---|---|---|---|---|---|---|---|
| cT1 | 230 | (7.3%) | 2918 | (92.7%) | 3148 | (64.0%) | p < 0.001 |
| cT2 | 219 | (14.4%) | 1306 | (85.6%) | 1525 | (31.0%) | |
| cT3 | 46 | (18.5%) | 203 | (81.5%) | 249 | (5.1%) | |

Lymph node stage

| | | | | | | | |
|---|---|---|---|---|---|---|---|
| cN0 | 340 | (9.0%) | 3433 | (91.0%) | 3773 | (80.6%) | p < 0.001 |
| cN1 | 123 | (15.2%) | 687 | (84.8%) | 810 | (17.3%) | |
| cN2 | 19 | (26.4%) | 53 | (73.6%) | 72 | (1.5%) | |
| cN3 | 11 | (37.9%) | 18 | (62.1%) | 29 | (0.6%) | |

Clinical subtype

| | | | | | | | |
|---|---|---|---|---|---|---|---|
| ER+/HER2+ | 47 | (12.6%) | 326 | (87.4%) | 373 | (4.9%) | p < 0.001 |
| ER+/HER2− | 301 | (4.6%) | 6228 | (95.4%) | 6529 | (86.5%) | |
| ER−/HER2+ | 44 | (37.9%) | 72 | (62.1%) | 116 | (1.5%) | |
| ER−/HER2− | 300 | (57.0%) | 226 | (43.0%) | 526 | (7.0%) | |

MammaPrint

| | | | | | | | |
|---|---|---|---|---|---|---|---|
| Low Risk | 16 | (0.3%) | 4876 | (99.7%) | 4892 | (48.8%) | p < 0.001 |
| High 1 | 179 | (4.9%) | 3454 | (95.1%) | 3633 | (36.3%) | |
| High 2 | 721 | (48.2%) | 775 | (51.8%) | 1496 | (14.9%) | |

BluePrint

| | | | | | | | |
|---|---|---|---|---|---|---|---|
| Luminal-Type | 222 | (2.5%) | 8549 | (97.5%) | 8771 | (87.5%) | p < 0.001 |
| HER2-Type | 81 | (21.5%) | 296 | (78.5%) | 377 | (3.8%) | |
| Basal-Type | 613 | (70.2%) | 260 | (29.8%) | 873 | (8.7%) | |

What is claimed is:

1. A method of typing a sample comprising breast cancer cells or comprising gene expression products from breast cancer cells, of an individual with breast cancer for predicting a response to auxiliary immune therapy, comprising:
(i) isolating RNA from the sample obtained from the individual;
(ii) determining an expression level of at least 16 marker genes to thereby provide an expression profile for the marker genes, wherein the marker genes are selected from the genes listed in Table 1;
(iii) comparing the individual's expression profile to a reference expression profile of the at least 16 marker genes;
thereby typing the sample for predicting a response to auxiliary immune therapy.

2. The method according to claim 1, wherein the sample is either a fresh frozen or a formalin-fixed paraffin-embedded sample.

3. The method according to claim 1, wherein the auxiliary immune therapy is treatment with an immune checkpoint inhibitor.

4. The method according to claim 1, wherein the auxiliary immune therapy comprises treatment with an immune checkpoint inhibitor.

5. The method according to claim 1, wherein the determination of the expression profile is performed using RNA-sequencing or microarray gene expression analysis.

6. The method according to claim 1, wherein the expression profile comprises at least 17 different marker genes wherein the marker genes are selected from the genes listed in Table 1.

7. The method according to claim 1, wherein the expression profile of all 53 different marker genes listed in Table 1 is determined.

8. The method according to claim 1, wherein the reference expression profile is composed of the average expression levels of the marker genes specified in step (ii) of individuals having a positive response to auxiliary immune therapy; of individuals not having a positive response to auxiliary immune therapy; or of a mixture of individuals having a positive response to auxiliary immune therapy and individuals not having a positive response to auxiliary immune therapy.

9. The method according to claim 1, wherein the individual's expression profile is compared to two reference expression profiles, wherein one reference expression profile is composed of the average expression levels of the marker genes specified in step (ii) of individuals having a positive response to auxiliary immune therapy and the other reference expression profile is composed of the average expression level of the marker genes specified in step (ii) of individuals not having a positive response to auxiliary immune therapy.

10. The method according to claim 1, wherein the response is a pathologic complete response (pCR).

11. A method of treating an individual with breast cancer, comprising
typing of a sample from said individual using a method according to claim 1;
treating the individual that is typed as predictive of having response to auxiliary immunotherapy with auxiliary immunotherapy, optionally in combination with chemotherapy; and
treating the individual that is typed as predictive of not having response to auxiliary immunotherapy with chemotherapy.

12. The method according to claim 11, wherein the auxiliary immunotherapy comprises an immune checkpoint inhibitor.

13. The method according to claim 12, wherein the immune checkpoint inhibitor is a programmed cell death protein 1 (PD1) or programmed cell death ligand 1 (PDL1) inhibitor.


SEQUENCE LISTING (53 sequences)

SEQ ID NO: 1 - DNA, 60 bp, synthetic construct
  source 1..60: mol_type = other DNA
accatgttgactttcctcatgtgtttccttatgactcagtaagttggcaaggtcctgact

SEQ ID NO: 2 - DNA, 60 bp, synthetic construct
  source 1..60: mol_type = other DNA
cttgggtcattgacctctcagggcctggcaggccagtgtctgggtttttcttgtggtgta

SEQ ID NO: 3 - DNA, 60 bp, synthetic construct
  source 1..60: mol_type = other DNA
aaccatgaaacgctactaactacaggaagcaaactaagcccccgctgtaatgaaacacct

SEQ ID NO: 4 - DNA, 60 bp, synthetic construct
  source 1..60: mol_type = other DNA
aagctgtgcctcgacacatcctcatcccaagcatgggacacctcaagatgaataataatt

SEQ ID NO: 5 - DNA, 60 bp, synthetic construct
  source 1..60: mol_type = other DNA
acatcgtgaggaacttccactaccttctcatcaaggatggctgcagggtgcctgctgtag

SEQ ID NO: 6 - DNA, 60 bp, synthetic construct
  source 1..60: mol_type = other DNA
acttgaatgtccaacgcaaagcaatacatgaactcatccaagtgatggctgaactgtcgc

SEQ ID NO: 7 - DNA, 60 bp, synthetic construct
  source 1..60: mol_type = other DNA
agaagccattaactacgggacattcaccatcaagtcagatgtgtggtcttttgggatcct

SEQ ID NO: 8 - DNA, 60 bp, synthetic construct
  source 1..60: mol_type = other DNA
caaagatgcatttacctctgtatcaactcaggaaatctcataagctggtaccactcagga

SEQ ID NO: 9 - DNA, 60 bp, synthetic construct
  source 1..60: mol_type = other DNA
caagaggcaaaggaatccatgtagtagatatcctctgcttaaaaactcactacggaggag

SEQ ID NO: 10 - DNA, 60 bp, synthetic construct
  source 1..60: mol_type = other DNA
ccattttaaagatggctacttaggaccatatggatgttgtactgatgtcatttgaccacg

SEQ ID NO: 11 - DNA, 60 bp, synthetic construct
  source 1..60: mol_type = other DNA
cttccttgtaaatatgatttatacctaactgaataaaaagctgttctgtcttcccaccca

SEQ ID NO: 12 - DNA, 60 bp, synthetic construct
  source 1..60: mol_type = other DNA
ctttcctgtttgcattggaagccgtggttatctctgttggctccatgggattgattatca

SEQ ID NO: 13 - DNA, 60 bp, synthetic construct
  source 1..60: mol_type = other DNA
gctgggacaagggatcccccttccctgtggttctattatattataattataattaaatat

SEQ ID NO: 14 - DNA, 60 bp, synthetic construct
  source 1..60: mol_type = other DNA
ggtgaggggaccctactttgttatcccaagtgctcttattctggtgagaagaaccttaat

SEQ ID NO: 15 - DNA, 60 bp, synthetic construct
  source 1..60: mol_type = other DNA
gtcaagccataattgttcttagtttgcagttacactaaaaggtgaccaatgatggtcacc

SEQ ID NO: 16 - DNA, 60 bp, synthetic construct
  source 1..60: mol_type = other DNA
gtgtgtaggacggggaggtcacgatggcgcgacgtctgcagaaatttcatgaggaggtat

SEQ ID NO: 17 - DNA, 60 bp, synthetic construct
  source 1..60: mol_type = other DNA
tacatgccagtgacacttccagtcccctttgtattccttaaataaactcaatgagctctt

SEQ ID NO: 18 - DNA, 60 bp, synthetic construct
  source 1..60: mol_type = other DNA
tcactcaggacttctctcctgaagaacacgcagtgctaaaactgaggatgatttccctaa

SEQ ID NO: 19 - DNA, 60 bp, synthetic construct
  source 1..60: mol_type = other DNA
tcagaaggctcttctgactactgggcaaagagtgtagatcagagcagcagtgaaaacaat

SEQ ID NO: 20 - DNA, 60 bp, synthetic construct
  source 1..60: mol_type = other DNA
tccaggatgagttacttgaaatttgccttgagtgtgttacctcctttccaagctcctcgt

SEQ ID NO: 21 - DNA, 60 bp, synthetic construct
  source 1..60: mol_type = other DNA
tgaaaaatcctgaggattcatcttgcacatctgagatctgagccagtcgctgtggttgtt

SEQ ID NO: 22 - DNA, 60 bp, synthetic construct
  source 1..60: mol_type = other DNA
tggaatactcacgtgagggaacttactttggccagcattgaccttcaaagtcagatggaa

SEQ ID NO: 23 - DNA, 60 bp, synthetic construct
  source 1..60: mol_type = other DNA
tggctattatttgacttgttgctggtttgggagtttatttgagtattgctgatctttttc

SEQ ID NO: 24 - DNA, 60 bp, synthetic construct
  source 1..60: mol_type = other DNA
tgtgaagcaaggttgtgtcttgcgcatggttaaaaataaagcattgtcctgcttcctaag

SEQ ID NO: 25 - DNA, 60 bp, synthetic construct
  source 1..60: mol_type = other DNA
ttgaaccctacacgaagaaagaactttctgctgttactttccctgacatcattcgcaatt

SEQ ID NO: 26 - DNA, 60 bp, synthetic construct
  source 1..60: mol_type = other DNA
ttttctgggaaatgacttttctgggaaatgacagtttctttgacatattttctttgccca

SEQ ID NO: 27 - DNA, 60 bp, synthetic construct
  source 1..60: mol_type = other DNA
agagtttcctttccctcttggccatattctggtgtcaatgacaaggagtaccttggcttt

SEQ ID NO: 28 - DNA, 60 bp, synthetic construct
  source 1..60: mol_type = other DNA
tgggactgttctgtctcatgtttatctgagctcttatctatgaagacatcttcccagagt

SEQ ID NO: 29 - DNA, 60 bp, synthetic construct
  source 1..60: mol_type = other DNA
agggcggccctgtactttttgtggcttggtttctctggaaatgtcaccttttcgggcgca

SEQ ID NO: 30 - DNA, 60 bp, synthetic construct
  source 1..60: mol_type = other DNA
aacaattacatttgtggtagcttggggttaccttcagctttcaaatacccacaggcacgc

SEQ ID NO: 31 - DNA, 60 bp, synthetic construct
  source 1..60: mol_type = other DNA
ccgctactacaaccagagcgaggcggggtatcacatcctccagggaatgtttggctgcga

SEQ ID NO: 32 - DNA, 60 bp, synthetic construct
  source 1..60: mol_type = other DNA
acatgactgcctatcagtagattgatctgtatttaatattcgttaattaaatctgcagtt

SEQ ID NO: 33 - DNA, 60 bp, synthetic construct
  source 1..60: mol_type = other DNA
agaagatagcacagatatcgggatattattgtgtgaaaatgctgcttttactttgatgtg

SEQ ID NO: 34 - DNA, 60 bp, synthetic construct
  source 1..60: mol_type = other DNA
aggctaggctggccatcagttgcttatttcagatgtgtcactaaattttccttctagatg

SEQ ID NO: 35 - DNA, 60 bp, synthetic construct
  source 1..60: mol_type = other DNA
aggctctgagactttactggcctatgcctgaggcctcttttcctttaactccctaaatta

SEQ ID NO: 36 - DNA, 60 bp, synthetic construct
  source 1..60: mol_type = other DNA
atacgcaaggggatgtggatacttggcccaaagtaactggtggtaggaatcttagaaaca

SEQ ID NO: 37 - DNA, 60 bp, synthetic construct
  source 1..60: mol_type = other DNA
atgcggaagatataaccttatatttggtaagtgtttcttgtgctattttatcacgtgacc

SEQ ID NO: 38 - DNA, 60 bp, synthetic construct
  source 1..60: mol_type = other DNA
atttcctgaacctgcgacggatgctggtgcagacacacctgcaggacctgaaagaggtga

SEQ ID NO: 39 - DNA, 60 bp, synthetic construct
  source 1..60: mol_type = other DNA
catcggaggccttaccactcctatgactcctgttttctctctcacagatagtgagggtct

SEQ ID NO: 40 - DNA, 60 bp, synthetic construct
  source 1..60: mol_type = other DNA
ccgaatgaccttaaaggtgatcggctttaacgaatatgtttacatatgcatagcgctgca

SEQ ID NO: 41 - DNA, 60 bp, synthetic construct
  source 1..60: mol_type = other DNA
ctcccatctgaaacctgtgactcaggtttatgaatggtgtttgtgtagcaacacattgtg

SEQ ID NO: 42 - DNA, 60 bp, synthetic construct
  source 1..60: mol_type = other DNA
ctgtcaaaagactctgtatatcttttgtggatgagttttgtgagagaacagagagaccat

SEQ ID NO: 43 - DNA, 60 bp, synthetic construct
  source 1..60: mol_type = other DNA
ggaaattggcgatgtcacccgtgtacggtacgcagcccagagcagaccctcaataaacgt

SEQ ID NO: 44 - DNA, 60 bp, synthetic construct
  source 1..60: mol_type = other DNA
ggaagcctgggagtgtttgtcccatcggtagcttgaaataaacgctcccctcagacaccc

SEQ ID NO: 45 - DNA, 60 bp, synthetic construct
  source 1..60: mol_type = other DNA
ggggaacagttcatactatagagagttacagtttagatgtatgtgtaaatcgattagcta

SEQ ID NO: 46 - DNA, 60 bp, synthetic construct
  source 1..60: mol_type = other DNA
taggttctatttatatggcagatgacgtgaaattgtgatgtttgttacagagcttttatg

SEQ ID NO: 47 - DNA, 60 bp, synthetic construct
  source 1..60: mol_type = other DNA
tccagccaaatagtgttctcggggtggtggctgggcagcgcctatgtttctctggagatt

SEQ ID NO: 48 - DNA, 60 bp, synthetic construct
  source 1..60: mol_type = other DNA
tcccctgaggaatatgtcatagttctgagctgccagtggaccgcccttttcccctaccaa

SEQ ID NO: 49 - DNA, 60 bp, synthetic construct
  source 1..60: mol_type = other DNA
tggcatatggactgaaagaaactatgctattggatctcctggatctccagcttgctgact

SEQ ID NO: 50 - DNA, 60 bp, synthetic construct
  source 1..60: mol_type = other DNA
tgggaaagaggactcacagttagccagctgaccatgactgaagttccagctttacttttt

SEQ ID NO: 51 - DNA, 60 bp, synthetic construct
  source 1..60: mol_type = other DNA
tgtcagggcgtgggacatctagtaggtgcttgacataatttcactgaattaatgacagag

SEQ ID NO: 52 - DNA, 60 bp, synthetic construct
  source 1..60: mol_type = other DNA
tttgcagaataattacactatcttatgtctggatcctgatgatttcacagctaaatggca

SEQ ID NO: 53 - DNA, 60 bp, synthetic construct
  source 1..60: mol_type = other DNA
ttttgtattgattttcacagctttgaggaacatgcataagaaatgtagctgaagtagagg